(19) United States
(12) Patent Application Publication
(10) Pub. No.: US 2016/0296648 A1
(43) Pub. Date: Oct. 13, 2016

(54) Y-90-LABELED ANTI-CD22 ANTIBODY (EPRATUZUMAB TETRAXETAN) IN REFRACTORY/RELAPSED ADULT CD22+ B-CELL ACUTE LYMPHOBLASTIC LEUKEMIA

(71) Applicants: Immunomedics, Inc., Morris Plains, NJ (US); CHU de Nantes, Nantes (FR); INSERM, Paris (FR); University of Nantes, Nantes (FR)

(72) Inventors: Patrice Chevallier, Nantes (FR); Francoise Kraeber-Bodere, Nantes (FR); David M. Goldenberg, Mendham, NJ (US)

(21) Appl. No.: 15/088,491

(22) Filed: Apr. 1, 2016

Related U.S. Application Data
(60) Provisional application No. 62/144,000, filed on Apr. 7, 2015.

(51) Int. Cl. A61K 51/10; C07K 16/28

(52) U.S. Cl. CPC A61K 51/1069; C07K 16/2803; A61K 51/1096; C07K 2317/565; C07K 2317/24

(57) ABSTRACT

The present invention relates to use of ⁹⁰Y-conjugated anti-CD22 antibody for treatment of relapsed/refractory acute lymphoblastic leukemia (ALL). Preferably the anti-CD22 antibody is epratuzumab tetraxetan. More preferably, the radiolabeled antibody is administered at a dosage of between 2.5 and 10.0 mCi/m², most preferably on days 1 and 8 of the cycle. In specific embodiments, the dosage may be 2.5, 5.0, 7.5 or 10.0 mCi/m². The radiolabeled antibody is capable of inducing a complete response in individuals with relapsed/refractory ALL.

### RELATED APPLICATIONS

This application claims the benefit under 35 U.S.C. 119(e) of U.S. Provisional Patent Application 62/144,000, filed Apr. 7, 2015, the text of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Mar. 21, 2016, is named IMM357US1_SL and is 13,980 bytes in size.

### FIELD OF THE INVENTION

The present invention relates to therapeutic use of conjugates of anti-CD22 antibodies with therapeutic radionuclides. In preferred embodiments, the anti-CD22 antibody is epratuzumab (also known as hLL2, see, e.g., U.S. Pat. Nos. 5,789,554 and 6,187,287) and the radionuclide is ⁹⁰Y. The conjugated antibody is of use to treat B-cell leukemias or lymphomas, particularly those that have relapsed from or are refractory to other standard anti-cancer therapies. In a particularly preferred embodiment, the cancer is relapsed/refractory acute lymphoblastic leukemia (ALL). Other embodiments relate to specific dosages and/or treatment cycles found to be of particular use to treat human ALL. A particularly preferred embodiment relates to use of a dosage of 2×10.0 mCi/m² one week apart, on a weekly cycle. The subject methods and compositions have been found to exhibit unexpectedly high efficacy and low toxicity for treating relapsed/refractory ALL.

### BACKGROUND OF THE INVENTION

The prognosis of relapsed/refractory acute lymphoblastic leukemia (ALL) in adults is dismal. The development of new therapies is needed in this setting, primarily in order to increase the number of patients who achieve a complete response and are thus eligible for allogeneic stem cell transplantion (all-SCT) (Thomas et al., 1999, Cancer 86:1216-30; Tavernier et al., 2007, Leukemia 21:1907-14; Fielding et al., 2007, Blood 109:944-50; Oriol et al., 2010, Haematologica 95:589-96; Gokbuget et al., 2012 Blood 120:2032-41). Targeted therapies are increasingly becoming treatment options for many hematological diseases. Our particular interest has been in radioimmunotherapy (RAIT).

Antibody-labeling with yttrium-90 (⁹⁰Y) could significantly increase the anti-tumor response by selective irradiation of tumor cells and their environment (Juweid et al., 2002, J Nucl Med 43:1507-29). Yttrium-90 (high-energy beta particle) by its metallic nature is well retained in the target cells after internalization and might be effective against cancer cells (Stein et al., 1999, Cancer Biother Radiopharm 14:37-47). Anti-CD20 RAIT with ⁹⁰Y-ibritumomab tiuxetan has been reported to be an effective treatment in indolent, B-cell, non-Hodgkin lymphoma (NHL), and is under investigation for aggressive NHL as part of conditioning regimens before allo-SCT (Sharkey & Goldenberg, 2011, Immunotherapy 3:349-70). RAIT has also been studied in acute myeloid leukemia using radiolabeled antibodies targeting CD45, CD66 or CD33 (Burke et al., 2002, Cancer Control 9:106-13). While immuno/chemoimmunotherapy is a recent area of active research in ALL (Hoelzer et al., 2012, Blood Reviews 26:25-32, we are unaware of any published studies in this setting using RAIT.

Of several surface antigens considered, CD22 is highly expressed in B-ALL (Raponi et al., 2011, Leukemia & Lymphoma 52:1098-1107). As such, the anti-CD22 humanized antibody, epratuzumab (Immunomedics, Inc., Morris Plains, N.J.), extensively studied in NHL (Leonard et al., 2004, Clin Cancer Res 10:5327-34; Micallef et al., 2011, Blood 118:4053-61), is also under active investigation in pediatric and adult ALL (Raetz et al., 2008, J Clin Oncol 26:3756-62; Advani et al., 2014, Br J Haematol 165:504-9). Epratuzumab acts through antibody-dependent cellular cytotoxicity, CD22 phosphorylation and proliferation inhibition following cross linking (Carnahan et al., 2007, Mol Immunol 44:1331-41). Anti-CD22 RAIT has been studied in NHL (Morschhauser et al., 2010, J Clin Oncol 28:3709-16; Kraeber-Bodere et al., 2012, Blood (ASH Annual Meeting Abstracts) 120:Abstract 906). A need exists for improved methods of administering ⁹⁰Y-DOTA-epratuzumab RAIT in adults with refractory/relapsed CD22⁺ B-ALL.

### SUMMARY OF THE INVENTION

### Definitions

The following definitions are provided to facilitate understanding of the disclosure herein. Where a term is not specifically defined, it is used in accordance with its plain and ordinary meaning.

As used herein, the terms “a”, “an” and “the” may refer to either the singular or plural, unless the context otherwise makes clear that only the singular is meant.

An “antibody” refers to a full-length (i.e., naturally occurring or formed by normal immunoglobulin gene fragment recombinatorial processes) immunoglobulin molecule (e.g., an IgG antibody).

An “antibody fragment” is a portion of an antibody such as F(ab′)₂, F(ab)₂, Fab′, Fab, Fv, scFv, single domain antibodies (DABs or VHHs) and the like, including half-molecules of IgG4 (van der Neut Kolfschoten et al., 2007, Science 317:1554-1557). Regardless of structure, an antibody fragment binds with the same antigen that is recognized by the intact antibody. For example, an anti-CD22 antibody fragment binds with an epitope of CD22. The term “antibody fragment” also includes isolated fragments consisting of the variable regions, such as the “Fv” fragments consisting of the variable regions of the heavy and light chains, recombinant single chain polypeptide molecules in which light and heavy chain variable regions are connected by a peptide linker (“scFv proteins”), and minimal recognition units consisting of the amino acid residues that mimic the hypervariable region.

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains, including human framework region (FR) sequences. The constant domains of the antibody molecule are derived from those of a human antibody.

A “human antibody” is an antibody obtained from transgenic mice that have been genetically engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. (See, e.g., McCafferty et al., Nature 348:552-553 (1990) for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

A “therapeutic agent” is an atom, molecule, or compound that is useful in the treatment of a disease. Examples of therapeutic agents include but are not limited to antibodies, antibody fragments, drugs, cytokine or chemokine inhibitors, pro-apoptotic agents, tyrosine kinase inhibitors, toxins, enzymes, nucleases, hormones, immunomodulators, antisense oligonucleotides, siRNA, RNAi, chelators, boron compounds, photoactive agents, dyes and radioisotopes.

A “diagnostic agent” is an atom, molecule, or compound that is useful in diagnosing a disease. Useful diagnostic agents include, but are not limited to, radioisotopes, dyes, contrast agents, fluorescent compounds or molecules and enhancing agents (e.g., paramagnetic ions). Preferably, the diagnostic agents are selected from the group consisting of radioisotopes, enhancing agents, and fluorescent compounds.

An “immunoconjugate” is a conjugate of an antibody with an atom, molecule, or a higher-ordered structure (e.g., with a liposome), a therapeutic agent, or a diagnostic agent.

A “naked antibody” is generally an entire antibody that is not conjugated to a therapeutic agent. This is so because the Fc portion of the antibody molecule provides effector functions, such as complement fixation and ADCC (antibody dependent cell cytotoxicity) that set mechanisms into action that may result in cell lysis. However, it is possible that the Fc portion is not required for therapeutic function, with other mechanisms, such as apoptosis, coming into play. Naked antibodies include both polyclonal and monoclonal antibodies, as well as certain recombinant antibodies, such as chimeric, humanized or human antibodies.

As used herein, the term “antibody fusion protein” is a recombinantly produced antigen-binding molecule in which an antibody or antibody fragment is linked to another protein or peptide, such as the same or different antibody or antibody fragment or a DDD or AD peptide (of the DOCK-AND-LOCK® complexes described below). The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators and toxins. One preferred toxin comprises a ribonuclease (RNase), preferably a recombinant RNase.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody, e.g., an IgG, is bivalent because it has two binding arms but is monospecific because it binds to one epitope. Multispecific, multivalent antibodies are constructs that have more than one binding site of different specificity.

A “bispecific antibody” is an antibody that can bind simultaneously to two targets which are of different structure. Bispecific antibodies (bsAb) and bispecific antibody fragments (bsFab) may have at least one arm that specifically binds to, for example, a B cell, T cell, myeloid-, plasma-, and mast-cell antigen or epitope and at least one other arm that specifically binds to a targetable conjugate that bears a therapeutic or diagnostic agent. A variety of bispecific antibodies can be produced using molecular engineering. Included herein are bispecific antibodies that target a cancer-associated antigen and also an immunotherapeutic T cell, such as CD3-T cells.

The term “direct cytotoxicity” refers to the ability of an agent to inhibit the proliferation or induce the apoptosis of a cell grown in an optimized culture medium in which only the agent and the cell are present.

### BRIEF DESCRIPTION OF THE DRAWINGS

The following drawings are provided to illustrate preferred embodiments of the invention. However, the claimed subject matter is in no way limited by the illustrative embodiments disclosed in the drawings.

FIG. 1A. Follow-up of immunophenotypic minimal residual disease (MRD) after radioimmunotherapy (RAIT) 1 & 2 in the bone marrow of a patient who achieved complete remission at level 2. Leukemic blasts were detected using an 8-color combination with a CD45(+/intermediate)/SSC(++)/CD19(+)/CD10(+)/CD34(neg)/CD38(++/dim)/CD58(++)/CD20 (+)/CD22(+) gating strategy.

FIG. 1B. Results from Day +90 to Day +270 post-RAIT

FIG. 2. Follow-up of molecular minimal residual disease (MRD) after radioimmunotherapy (RAIT) cycle 1 & 2 (arrows) in the bone marrow of the patient achieving complete remission at level 2. MRD is expressed as a percentage of the e1a2/p190 Bcr-Abl fusion transcript/Abl. The patient relapsed at 12 months.

FIG. 3. Examples of plasma pharmacokinetics of ⁹⁰Yttrium-labeled anti-CD22 epratuzumab tetraxetan in two patients.

FIG. 4. An example of favorable biodistribution of 90Yttrium-labeled anti-CD22 epratuzumab tetraxetan in one patient. SPECT-CT whole-body images (anterior projection) illustrate the biodistribution of ¹¹¹In-epratuzumab respectively at 4 hours (D0), 1 day (D1), 5 days (D5) and 7 days (D7) after infusion. Early images at 4 hours and 1 day demonstrate blood-pool activity in heart and large blood vessels (red arrows). Between day 1 and day 5, blood-pool activity faded, liver and spleen uptake decreased, while BM (spine, iliac bones, femurs and humeri) activity rose and persisted at day 7.

FIG. 5. Median organs absorbed doses of ⁹⁰Y-epratuzumab tetraxetan in mGy/MBq (n=11 patients measured by SPECT-CT. Abbreviations: WB: whole body; RL: right lung; LL: left lung, RK: right kidney; LK: left kidney; BM: bone marrow.

FIG. 6A. Correlation between Flt3-L concentration, hematologic toxicities and responses. Level 1 (92.5 MBq/m²): 4 patients out of 5 were tested. None of them showed Flt3-L increase after the RAIT, suggesting no hematologic toxicity of the procedure.

FIG. 6B. Correlation between Flt3-L concentration, hematologic toxicities and responses. Level 2 (185 MBq/m²): The three patients were tested. The two non-responders showed no Flt3-L increase after the RAIT. Patient 8 who achieved a CR (documented at day+32) showed an increased Flt3-L concentration as soon as 2 weeks after treatment initiation and a persistent high concentration at time of CR. Patient 8 received a second RAIT cycle, 8 and 9 weeks (**) after treatment initiation. Documentation of Flt3-L concentration increase after the second RAIT cycle was not possible in the responder because no samples were available early after the second cycle. However in that patient, the first sample available 4 weeks after the second RAIT did not show significant increase of Flt3-L concentration.

FIG. 6C. Correlation between Flt3-L concentration, hematologic toxicities and responses. Level 3 (277, 5 MBq/m²): Only one out of three patients was tested. No Flt3-L increase was observed after the RAIT.

FIG. 6D. Correlation between Flt3-L concentration, hematologic toxicities and responses. Level 4 (370 MBq/m²): Two patients out of 6 were tested, corresponding to the two responders.

### MONOCLONAL ANTIBODIES

The compositions, formulations and methods described herein may include monoclonal antibodies. Rodent monoclonal antibodies to specific antigens may be obtained by methods known to those skilled in the art. (See, e.g., Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991)). General techniques for cloning murine immunoglobulin variable domains have been disclosed, for example, by the publication of Orlandi et al., Proc. Nat'l Acad. Sci. USA 86: 3833 (1989).

Chimeric Antibodies

A chimeric antibody is a recombinant protein that contains the variable domains including the CDRs derived from one species of animal, such as a rodent antibody, while the remainder of the antibody molecule; i.e., the constant domains, is derived from a human antibody. Techniques for constructing chimeric antibodies are well known to those of skill in the art. As an example, Leung et al., Hybridoma 13:469 (1994), disclose how they produced an LL2 chimera by combining DNA sequences encoding the Vₖ and V_{H} domains of LL2 monoclonal antibody, an anti-CD22 antibody, with respective human and IgG₁ constant region domains. This publication also provides the nucleotide sequences of the LL2 light and heavy chain variable regions, Vₖ and V_{H}, respectively.

Humanized Antibodies

A chimeric monoclonal antibody can be humanized by replacing the sequences of the murine FR in the variable domains of the chimeric antibody with one or more different human FR. Specifically, mouse CDRs are transferred from heavy and light variable chains of the mouse immunoglobulin into the corresponding variable domains of a human antibody. As simply transferring mouse CDRs into human FRs often results in a reduction or even loss of antibody affinity, additional modification might be required in order to restore the original affinity of the murine antibody. This can be accomplished by the replacement of one or more some human residues in the FR regions with their murine counterparts to obtain an antibody that possesses good binding affinity to its epitope. (See, e.g., Tempest et al., Biotechnology 9:266 (1991) and Verhoeyen et al., Science 239: 1534 (1988)). Techniques for producing humanized antibodies are disclosed, for example, by Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993).

Human Antibodies

A fully human antibody can be obtained from a transgenic non-human animal. (See, e.g., Mendez et al., Nature Genetics, 15: 146-156, 1997; U.S. Pat. No. 5,633,425.) Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Pharmacol. 3:544-50; each incorporated herein by reference). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40, incorporated herein by reference). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.). Recombinant Fab were cloned from the μ, γ and κ chain antibody repertoires and inserted into a phage display library (Id.). RNAs were converted to cDNAs and used to make Fab cDNA libraries using specific primers against the heavy and light chain immunoglobulin sequences (Marks et al., 1991, J Mol. Biol. 222:581-97). Library construction was performed according to Andris-Widhopf et al. (2000, In: Phage Display Laboratory Manual, Barbas et al. (eds), edition, Cold Spring Harbor Laboratory Press, Cold Spring Harbor, N.Y. pp. 9.1 to 9.22, incorporated herein by reference). The final Fab fragments were digested with restriction endonucleases and inserted into the bacteriophage genome to make the phage display library. Such libraries may be screened by standard phage display methods. The skilled artisan will realize that this technique is exemplary only and any known method for making and screening human antibodies or antibody fragments by phage display may be utilized.

In another alternative, transgenic animals that have been genetically engineered to produce human antibodies may be used to generate antibodies against essentially any immunogenic target, using standard immunization protocols as discussed above. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A non-limiting example of such a system is the XenoMouse® (e.g., Green et al., 1999, J. Immunol. Methods 231:11-23, incorporated herein by reference) from Abgenix (Fremont, Calif.). In the XenoMouse® and similar animals, the mouse antibody genes have been inactivated and replaced by functional human antibody genes, while the remainder of the mouse immune system remains intact.

The XENOMOUSE® was transformed with germline-configured YACs (yeast artificial chromosomes) that contained portions of the human IgH and Ig kappa loci, including the majority of the variable region sequences, along accessory genes and regulatory sequences. The human variable region repertoire may be used to generate antibody producing B cells, which may be processed into hybridomas by known techniques. After immunization with a target antigen, the engineered mice will produce human antibodies by the normal immune response, which may be harvested and/or produced by standard techniques discussed above. A variety of strains of engineered mice are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the mouse system but may utilize any transgenic animal that has been genetically engineered to produce human antibodies.

Antibody Cloning and Production

Various techniques, such as production of chimeric or humanized antibodies, may involve procedures of antibody cloning and construction. The antigen-binding V_{κ} (variable light chain) and V_{H} (variable heavy chain) sequences for an antibody of interest may be obtained by a variety of molecular cloning procedures, such as RT-PCR, 5′-RACE, and cDNA library screening. The V genes of an antibody from a cell that expresses a murine antibody can be cloned by PCR amplification and sequenced. To confirm their authenticity, the cloned V_{L} and V_{H} genes can be expressed in cell culture as a chimeric Ab as described by Orlandi et al., (Proc. Natl. Acad. Sci., USA, 86: 3833 (1989)). Based on the V gene sequences, a humanized antibody can then be designed and constructed as described by Leung et al. (Mol. Immunol., 32: 1413 (1995)).

cDNA can be prepared from any known hybridoma line or transfected cell line producing a murine antibody by general molecular cloning techniques (Sambrook et al., Molecular Cloning, A laboratory manual, 2^{nd} Ed (1989)). The V_{κ} sequence for the antibody may be amplified using the primers VK1BACK and VK1FOR (Orlandi et al., 1989) or the extended primer set described by Leung et al. (BioTechniques, 15: 286 (1993)). The V_{H} sequences can be amplified using the primer pair VH1BACK/VH1FOR (Orlandi et al., 1989) or the primers annealing to the constant region of murine IgG described by Leung et al. (Hybridoma, 13:469 (1994)). Humanized V genes can be constructed by a combination of long oligonucleotide template syntheses and PCR amplification as described by Leung et al. (Mol. Immunol., 32: 1413 (1995)).

PCR products for V_{κ} can be subcloned into a staging vector, such as a pBR327-based staging vector, VKpBR, that contains an Ig promoter, a signal peptide sequence and convenient restriction sites. PCR products for V_{H} can be subcloned into a similar staging vector, such as the pBluescript-based VHpBS. Expression cassettes containing the V_{κ} and V_{H} sequences together with the promoter and signal peptide sequences can be excised from VKpBR and VHpBS and ligated into appropriate expression vectors, such as pKh and pG1g, respectively (Leung et al., Hybridoma, 13:469 (1994)). The expression vectors can be co-transfected into an appropriate cell and supernatant fluids monitored for production of a chimeric, humanized or human antibody. Alternatively, the V_{κ} and V_{H} expression cassettes can be excised and subcloned into a single expression vector, such as pdHL2, as described by Gillies et al. Immunol. Methods 125:191 (1989) and also shown in Losman et al., Cancer, 80:2660 (1997)).

In an alternative embodiment, expression vectors may be transfected into host cells that have been pre-adapted for transfection, growth and expression in serum-free medium. Exemplary cell lines that may be used include the Sp/EEE, Sp/ESF and Sp/ESF-X cell lines (see, e.g., U.S. Pat. Nos. 7,531,327; 7,537,930 and 7,608,425; the Examples section of each of which is incorporated herein by reference). These exemplary cell lines are based on the Sp2/0 myeloma cell line, transfected with a mutant Bcl-EEE gene, exposed to methotrexate to amplify transfected gene sequences and pre-adapted to serum-free cell line for protein expression.

Antibody Allotypes

Immunogenicity of therapeutic antibodies is associated with increased risk of infusion reactions and decreased duration of therapeutic response (Baert et al., 2003, N Engl J Med 348:602-08). The extent to which therapeutic antibodies induce an immune response in the host may be determined in part by the allotype of the antibody (Stickler et al., 2011, Genes and Immunity 12:213-21). Antibody allotype is related to amino acid sequence variations at specific locations in the constant region sequences of the antibody. The allotypes of IgG antibodies containing a heavy chain γ-type constant region are designated as Gm allotypes (1976, J Immunol 117:1056-59).

For the common IgG1 human antibodies, the most prevalent allotype is G1m1 (Stickler et al., 2011, Genes and Immunity 12:213-21). However, the G1m3 allotype also occurs frequently in Caucasians (Id.). It has been reported that Glm1 antibodies contain allotypic sequences that tend to induce an immune response when administered to non-Glm1 (nG1m1) recipients, such as G1m3 patients (Id.). Non-Glm1 allotype antibodies are not as immunogenic when administered to Glm1 patients (Id.).

The human Glm1 allotype comprises the amino acids aspartic acid at Kabat position 356 and leucine at Kabat position 358 in the CH3 sequence of the heavy chain IgG1. The nGlm1 allotype comprises the amino acids glutamic acid at Kabat position 356 and methionine at Kabat position 358. Both Glm1 and nGlm1 allotypes comprise a glutamic acid residue at Kabat position 357 and the allotypes are sometimes referred to as DEL and EEM allotypes. A non-limiting example of the heavy chain constant region sequences for Glm1 and nGlm1 allotype antibodies is shown for the exemplary antibodies rituximab (SEQ ID NO:9) and veltuzumab (SEQ ID NO:8).

Veltuzumab heavy chain constant region sequence
(SEQ ID NO: 8)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Rituximab heavy chain constant region sequence
(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

Jefferis and Lefranc (2009, mAbs 1:1-7) reviewed sequence variations characteristic of IgG allotypes and their effect on immunogenicity. They reported that the G1m3 allotype is characterized by an arginine residue at Kabat position 214, compared to a lysine residue at Kabat 214 in the G1m17 allotype. The nG1m1,2 allotype was characterized by glutamic acid at Kabat position 356, methionine at Kabat position 358 and alanine at Kabat position 431. The G1m1,2 allotype was characterized by aspartic acid at Kabat position 356, leucine at Kabat position 358 and glycine at Kabat position 431. In addition to heavy chain constant region sequence variants, Jefferis and Lefranc (2009) reported allotypic variants in the kappa light chain constant region, with the Km1 allotype characterized by valine at Kabat position 153 and leucine at Kabat position 191, the Km1,2 allotype by alanine at Kabat position 153 and leucine at Kabat position 191, and the Km3 allotype characterized by alanine at Kabat position 153 and valine at Kabat position 191.

With regard to therapeutic antibodies, veltuzumab and rituximab are, respectively, humanized and chimeric IgG1 antibodies against CD20, of use for therapy of a wide variety of hematological malignancies and/or autoimmune diseases. Table 1 compares the allotype sequences of rituximab vs. veltuzumab. As shown in Table 1, rituximab (G1m17,1) is a DEL allotype IgG1, with an additional sequence variation at Kabat position 214 (heavy chain CH1) of lysine in rituximab vs. arginine in veltuzumab. It has been reported that veltuzumab is less immunogenic in subjects than rituximab (see, e.g., Morchhauser et al., 2009, J Clin Oncol 27:3346-53; Goldenberg et al., 2009, Blood 113:1062-70; Robak & Robak, 2011, BioDrugs 25:13-25), an effect that has been attributed to the difference between humanized and chimeric antibodies. However, the difference in allotypes between the EEM and DEL allotypes likely also accounts for the lower immunogenicity of veltuzumab.

TABLE 1
Allotypes of Rituximab vs. Veltuzumab

| | | Heavy chain position and | | | | | |
|---|---|---|---|---|---|---|---|
| | | associated allotypes | | | | | |
| | | 214 | | 356/358 | | 431 | |
| | Complete allotype | (allotype) | | (allotype) | | (allotype) | |
| Rituximab | G1m17,1 | K | 17 | D/L | 1 | A | — |
| Veltuzumab | G1m3 | R | 3 | E/M | — | A | — |

In order to reduce the immunogenicity of therapeutic antibodies in individuals of nG1m1 genotype, it is desirable to select the allotype of the antibody to correspond to the G1m3 allotype, characterized by arginine at Kabat 214, and the nG1m1,2 null-allotype, characterized by glutamic acid at Kabat position 356, methionine at Kabat position 358 and alanine at Kabat position 431. Surprisingly, it was found that repeated subcutaneous administration of G1m3 antibodies over a long period of time did not result in a significant immune response. In alternative embodiments, the human IgG4 heavy chain in common with the G1m3 allotype has arginine at Kabat 214, glutamic acid at Kabat 356, methionine at Kabat 359 and alanine at Kabat 431. Since immunogenicity appears to relate at least in part to the residues at those locations, use of the human IgG4 heavy chain constant region sequence for therapeutic antibodies is also a preferred embodiment. Combinations of G1m3 IgG1 antibodies with IgG4 antibodies may also be of use for therapeutic administration.

Known Antibodies

In various embodiments, the claimed methods and compositions may utilize any of a variety of antibodies known in the art. For example, therapeutic use of radiolabeled anti-CD22 antibody may be supplemented with one or more antibodies against other disease-associated antigens. Antibodies of use may be commercially obtained from a number of known sources. For example, a variety of antibody secreting hybridoma lines are available from the American Type Culture Collection (ATCC, Manassas, Va.). A large number of antibodies against various disease targets, including but not limited to tumor-associated antigens, have been deposited at the ATCC and/or have published variable region sequences and are available for use in the claimed methods and compositions. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572,856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040; 6,451,310; 6,444,206; 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953, 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art (see, e.g., U.S. Pat. Nos. 7,531,327; 7,537,930; 7,608,425 and 7,785,880, the Examples section of each of which is incorporated herein by reference).

Antibodies of use may bind to various known antigens expressed in B cells or T cells, including but not limited to BCL-1, BCL-2, BCL-6, CD1a, CD2, CD3, CD4, CD5, CD7, CD8, CD10, CD11b, CD11c, CD13, CD14, CD15, CD16, CD19, CD20, CD21, CD22, CD23, CD25, CD33, CD34, CD38, CD40, CD40L, CD41a, CD43, CD45, CD47, CD55, CD56, CCD57, CD59, CD64, CD71, CD79a, CD79b, CD117, CD138, CXCR4, FMC-7 and HLA-DR.

Particular antibodies that may be of use for therapy of cancer within the scope of the claimed methods and compositions include, but are not limited to, LL1 (anti-CD74), LL2 and RFB4 (anti-CD22), RS7 (anti-epithelial glycoprotein-1 (EGP-1)), PAM4 and KC4 (both anti-mucin), MN-14 (anti-carcinoembryonic antigen (CEA, also known as CD66e), MN-15 (anti-CEACAM6), Mu-9 (anti-colon-specific antigen-p), Immu 31 (an anti-alpha-fetoprotein), TAG-72 (e.g., CC49), R1 (anti-IGF-1R), Tn, J591 or HuJ591 (anti-PSMA (prostate-specific membrane antigen), AB-PG1-XG1-026 (anti-PSMA dimer), D2/B (anti-PSMA), G250 (anti-carbonic anhydrase IX), hL243 (anti-HLA-DR), alemtuzumab (anti-CD52), bevacizumab (anti-VEGF), cetuximab (anti-EGFR), gemtuzumab (anti-CD33), ibritumomab (anti-CD20), panitumumab (anti-EGFR), rituximab (anti-CD20), tositumomab (anti-CD20), GA101 (anti-CD20; obinutuzumab) and trastuzumab (anti-ErbB2). Such antibodies are known in the art (e.g., U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20040202666 (now abandoned); 20050271671; and 20060193865; the Examples section of each incorporated herein by reference.) Specific known antibodies of use include hPAM4 (U.S. Pat. No. 7,282,567), hA20 (U.S. Pat. No. 7,251,164), hA19 (U.S. Pat. No. 7,109,304), hIMMU31 (U.S. Pat. No. 7,300,655), hLL1 (U.S. Pat. No. 7,312,318), hLL2 (U.S. Pat. No. 5,789,554), hMu-9 (U.S. Pat. No. 7,387,773), hL243 (U.S. Pat. No. 7,612,180), hMN-14 (U.S. Pat. No. 6,676,924), hMN-15 (U.S. Pat. No. 8,287,865), hR1 (U.S. patent application Ser. No. 12/772,645), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575), the text of each recited patent or application is incorporated herein by reference with respect to the Figures and Examples sections.

In a particularly preferred embodiment, an anti-CD22 antibody of use is an hLL2 antibody (also known as epratuzumab) (see, U.S. Pat. No. 5,789,554). For purposes of this application, an hLL2 antibody is one that comprises the light chain complementarity determining region (CDR) sequences CDR1 (KSSQSVLYSANHKYLA, SEQ ID NO:16), CDR2 (WASTRES, SEQ ID NO:17), and CDR3 (HQYLSSWTF, SEQ ID NO:18) and the heavy chain CDR sequences CDR1 (SYWLH, SEQ ID NO:19), CDR2 (YINPRNDYTEYNQNFKD, SEQ ID NO:20), and CDR3 (RDITTFY, SEQ ID NO:21).

Antibody Fragments

Antibody fragments which recognize specific epitopes can be generated by known techniques. The antibody fragments are antigen binding portions of an antibody, such as F(ab)₂, Fab′, Fab, Fv, scFv and the like. Other antibody fragments include, but are not limited to: the F(ab′)₂ fragments which can be produced by pepsin digestion of the antibody molecule and the Fab′ fragments, which can be generated by reducing disulfide bridges of the F(ab′)₂ fragments. Alternatively, Fab′ expression libraries can be constructed (Huse et al., 1989, Science, 246:1274-1281) to allow rapid and easy identification of monoclonal Fab′ fragments with the desired specificity. In certain embodiments, the antibody fragment may be a fragment that is not an scFv fragment.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are disclosed in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIBTECH, Vol 9: 132-137 (1991).

An antibody fragment can be prepared by known methods, for example, as disclosed by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

A single complementarity-determining region (CDR) is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. (See, e.g., Larrick et al., Methods: A Companion to Methods in Enzymology 2: 106 (1991); Courtenay-Luck, “Genetic Manipulation of Monoclonal Antibodies,” in MONOCLONAL ANTIBODIES: PRODUCTION, ENGINEERING AND CLINICAL APPLICATION, Ritter et al. (eds.), pages 166-179 (Cambridge University Press 1995); and Ward et al., “Genetic Manipulation and Expression of Antibodies,” in MONOCLONAL ANTIBODIES: PRINCIPLES AND APPLICATIONS, Birch et al., (eds.), pages 137-185 (Wiley-Liss, Inc. 1995).

Another form of an antibody fragment is a single-domain antibody (dAb), sometimes referred to as a single chain antibody. Techniques for producing single-domain antibodies are well known in the art (see, e.g., Cossins et al., Protein Expression and Purification, 2007, 51:253-59; Shuntao et al., Molec Immunol 2006, 43:1912-19; Tanha et al., J. Biol. Chem. 2001, 276:24774-780).

In certain embodiments, the sequences of antibodies, such as the Fc portions of antibodies, may be varied to optimize the physiological characteristics of the conjugates, such as the half-life in serum. Methods of substituting amino acid sequences in proteins are widely known in the art, such as by site-directed mutagenesis (e.g. Sambrook et al., Molecular Cloning, A laboratory manual, 2^{nd} Ed, 1989). In preferred embodiments, the variation may involve the addition or removal of one or more glycosylation sites in the Fc sequence (e.g., U.S. Pat. No. 6,254,868, the Examples section of which is incorporated herein by reference). In other preferred embodiments, specific amino acid substitutions in the Fc sequence may be made (e.g., Hornick et al., 2000, J Nucl Med 41:355-62; Hinton et al., 2006, J Immunol 176:346-56; Petkova et al. 2006, Int Immunol 18:1759-69; U.S. Pat. No. 7,217,797; Hwang and Foote, 2005, Methods 36:3-10; Clark, 2000, Immunol Today 21:397-402; J Immunol 1976 117:1056-60; Ellison et al., 1982, Nucl Acids Res 13:4071-79; Stickler et al., 2011, Genes and Immunity 12:213-21).

Multispecific and Multivalent Antibodies

Methods for producing bispecific antibodies include engineered recombinant antibodies which have additional cysteine residues so that they crosslink more strongly than the more common immunoglobulin isotypes. (See, e.g., FitzGerald et al, Protein Eng. 10(10):1221-1225, (1997)). Another approach is to engineer recombinant fusion proteins linking two or more different single-chain antibody or antibody fragment segments with the needed dual specificities. (See, e.g., Coloma et al., Nature Biotech. 15:159-163, (1997)). A variety of bispecific antibodies can be produced using molecular engineering. In one form, the bispecific antibody may consist of, for example, an scFv with a single binding site for one antigen and a Fab fragment with a single binding site for a second antigen. In another form, the bispecific antibody may consist of, for example, an IgG with two binding sites for one antigen and two scFv with two binding sites for a second antigen. In alternative embodiments, multispecific and/or multivalent antibodies may be produced as DOCK-AND-LOCK® (DNL®) complexes as described below.

In certain embodiments, a radiolabeled anti-CD22 antibody or fragment may be administered to a patient as part of a combination of antibodies. Bispecific antibodies are preferred to administration of combinations of separate antibodies, due to cost and convenience. However, where combinations of separate antibodies provide improved safety or efficacy, the combination may be utilized. The antibodies may bind to different epitopes of the same antigen or to different antigens. Preferably, the antigens are selected from the group consisting of BCL-1, BCL-2, BCL-6, CD1a, CD2, CD3, CD4, CD5, CD7, CD8, CD10, CD11b, CD11c, CD13, CD14, CD15, CD16, CD19, CD20, CD21, CD22, CD23, CD25, CD33, CD34, CD38, CD40, CD40L, CD41a, CD43, CD45, CD47, CD55, CD56, CCD57, CD59, CD64, CD71, CD79a, CD79b, CD117, CD138, CXCR4, FMC-7 and HLA-DR. However, antibodies against other antigens of use for therapy of cancer, autoimmune diseases or immune dysfunction diseases are known in the art, as discussed below, and antibodies against any such disease-associated antigen known in the art may be utilized.

DOCK-AND-LOCK® (DNL®)

In preferred embodiments, a bivalent or multivalent antibody is formed as a DOCK-AND-LOCK® (DNL®) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264. Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Although the standard DNL® complex comprises a trimer with two DDD-linked molecules attached to one AD-linked molecule, variations in complex structure allow the formation of dimers, trimers, tetramers, pentamers, hexamers and other multimers. In some embodiments, the DNL® complex may comprise two or more antibodies, antibody fragments or fusion proteins which bind to the same antigenic determinant or to two or more different antigens. The DNL® complex may also comprise one or more other effectors, such as proteins, peptides, immunomodulators, cytokines, interleukins, interferons, binding proteins, peptide ligands, carrier proteins, toxins, ribonucleases such as onconase, inhibitory oligonucleotides such as siRNA, antigens or xenoantigens, polymers such as PEG, enzymes, therapeutic agents, hormones, cytotoxic agents, anti-angiogenic agents, pro-apoptotic agents or any other molecule or aggregate.

PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968; 243:3763). The structure of the holoenzyme consists of two catalytic subunits held in an inactive form by the R subunits (Taylor, J. Biol. Chem. 1989; 264:8443). Isozymes of PKA are found with two types of R subunits (RI and RII), and each type has α and β isoforms (Scott, Pharmacol. Ther. 1991; 50:123). Thus, the four isoforms of PKA regulatory subunits are RIα, RIβ, RIIα and RIIβ. The R subunits have been isolated only as stable dimers and the dimerization domain has been shown to consist of the first 44 amino-terminal residues of RIIα or RIIβ (Newlon et al., Nat. Struct. Biol. 1999; 6:222). As discussed below, similar portions of the amino acid sequences of other regulatory subunits are involved in dimerization and docking, each located at or near the N-terminal end of the regulatory subunit. Binding of cAMP to the R subunits leads to the release of active catalytic subunits for a broad spectrum of serine/threonine kinase activities, which are oriented toward selected substrates through the compartmentalization of PKA via its docking with AKAPs (Scott et al., J. Biol. Chem. 1990; 265; 21561)

Since the first AKAP, microtubule-associated protein-2, was characterized in 1984 (Lohmann et al., Proc. Natl. Acad. Sci USA. 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell Biol. 2004; 5:959). The AD of AKAPs for PKA is an amphipathic helix of 14-18 residues (Carr et al., J. Biol. Chem. 1991; 266:14188). The amino acid sequences of the AD are quite varied among individual AKAPs, with the binding affinities reported for RII dimers ranging from 2 to 90 nM (Alto et al., Proc. Natl. Acad. Sci. USA. 2003; 100:4445). AKAPs will only bind to dimeric R subunits. For human RIIα, the AD binds to a hydrophobic surface formed by the 23 amino-terminal residues (Colledge and Scott, Trends Cell Biol. 1999; 6:216). Thus, the dimerization domain and AKAP binding domain of human RIIα are both located within the same N-terminal 44 amino acid sequence (Newlon et al., Nat. Struct. Biol. 1999; 6:222; Newlon et al., EMBO J. 2001; 20:1651), which is termed the DDD herein.

We have developed a platform technology to utilize the DDD of human PKA regulatory subunits and the AD of AKAP as an excellent pair of linker modules for docking any two entities, referred to hereafter as A and B, into a noncovalent complex, which could be further locked into a DNL® complex through the introduction of cysteine residues into both the DDD and AD at strategic positions to facilitate the formation of disulfide bonds. The general methodology of the approach is as follows. Entity A is constructed by linking a DDD sequence to a precursor of A, resulting in a first component hereafter referred to as a. Because the DDD sequence would effect the spontaneous formation of a dimer, A would thus be composed of a₂. Entity B is constructed by linking an AD sequence to a precursor of B, resulting in a second component hereafter referred to as b. The dimeric motif of DDD contained in a₂ will create a docking site for binding to the AD sequence contained in b, thus facilitating a ready association of a₂ and b to form a binary, trimeric complex composed of a₂b. This binding event is made irreversible with a subsequent reaction to covalently secure the two entities via disulfide bridges, which occurs very efficiently based on the principle of effective local concentration because the initial binding interactions should bring the reactive thiol groups placed onto both the DDD and AD into proximity (Chmura et al., Proc. Natl. Acad. Sci. USA. 2001; 98:8480) to ligate site-specifically. Using various combinations of linkers, adaptor modules and precursors, a wide variety of DNL® constructs of different stoichiometry may be produced and used (see, e.g., U.S. Pat. Nos. 7,550,143; 7,521,056; 7,534,866; 7,527,787 and 7,666,400.)

By attaching the DDD and AD away from the functional groups of the two precursors, such site-specific ligations are also expected to preserve the original activities of the two precursors. This approach is modular in nature and potentially can be applied to link, site-specifically and covalently, a wide range of substances, including peptides, proteins, antibodies, antibody fragments, and other effector moieties with a wide range of activities. Utilizing the fusion protein method of constructing AD and DDD conjugated effectors described in the Examples below, virtually any protein or peptide may be incorporated into a DNL® construct. However, the technique is not limiting and other methods of conjugation may be utilized.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2^{nd} Ed, 1989). In such preferred embodiments, the AD and/or DDD moiety may be attached to either the N-terminal or C-terminal end of an effector protein or peptide. However, the skilled artisan will realize that the site of attachment of an AD or DDD moiety to an effector moiety may vary, depending on the chemical nature of the effector moiety and the part(s) of the effector moiety involved in its physiological activity. Site-specific attachment of a variety of effector moieties may be performed using techniques known in the art, such as the use of bivalent cross-linking reagents and/or other chemical conjugation techniques.

a. Structure-Function Relationships in AD and DDD Moieties

For different types of DNL® constructs, different AD or DDD sequences may be utilized. Exemplary DDD and AD sequences are provided below.

DDD1
(SEQ ID NO: 1)
SHIQIPPGLTELLQGYTVEVLRQQPPDLVEFAVEYFTRLREARA
DDD2
(SEQ ID NO: 2)
CGHIQIPPGLTELLQGYTVEVLRQQPPDLVEFAVEYFTRLREARA
AD1
(SEQ ID NO: 3)
QIEYLAKQIVDNAIQQA
AD2
(SEQ ID NO: 4)
CGQIEYLAKQIVDNAIQQAGC

The skilled artisan will realize that DDD1 and DDD2 are based on the DDD sequence of the human RIIα isoform of protein kinase A. However, in alternative embodiments, the DDD and AD moieties may be based on the DDD sequence of the human Ma form of protein kinase A and a corresponding AKAP sequence, as exemplified in DDD3, DDD3C and AD3 below.

DDD3
(SEQ ID NO: 5)
SLRECELYVQKHNIQALLKDSIVQLCTARPERPMAFLREYFERLEKEEAK
DDD3C
(SEQ ID NO: 6)
MSCGGSLRECELYVQKHNIQALLKDSIVQLCTARPERPMAFLREYFERLE
KEEAK
AD3
(SEQ ID NO: 7)
CGFEELAWKIAKMIWSDVFQQGC

In other alternative embodiments, other sequence variants of AD and/or DDD moieties may be utilized in construction of the DNL® complexes. For example, there are only four variants of human PKA DDD sequences, corresponding to the DDD moieties of PKA RIα, RIIα, RIβ and RIIβ. The RIIα DDD sequence is the basis of DDD1 and DDD2 disclosed above. The four human PKA DDD sequences are shown below. The DDD sequence represents residues 1-44 of RIIα, 1-44 of RIIβ, 12-61 of RIα and 13-66 of RIβ. (Note that the sequence of DDD1 is modified slightly from the human PKA RIIα DDD moiety.)

PKA RIα
(SEQ ID NO: 10)
SLRECELYVQKHNIQALLKDVSIVQLCTARPERPMAFLREYFEKLEKEEA
K
PKA RIβ
(SEQ ID NO: 11)
SLKGCELYVQLHGIQQVLKDCIVHLCISKPERPMKFLREHFEKLEKEENR
QILA
PKA RIIα
(SEQ ID NO: 12)
SHIQIPPGLTELLQGYTVEVGQQPPDLVDFAVEYFTRLREARRQ
PKA RIIβ
(SEQ ID NO: 13)
SIEIPAGLTELLQGFTVEVLRHQPADLLEFALQHFTRLQQENER

The structure-function relationships of the AD and DDD domains have been the subject of investigation. (See, e.g., Burns-Hamuro et al., 2005, Protein Sci 14:2982-92; Carr et al., 2001, J Biol Chem 276:17332-38; Alto et al., 2003, Proc Natl Acad Sci USA 100:4445-50; Hundsrucker et al., 2006, Biochem J 396:297-306; Stokka et al., 2006, Biochem J 400:493-99; Gold et al., 2006, Mol Cell 24:383-95; Kinderman et al., 2006, Mol Cell 24:397-408, the entire text of each of which is incorporated herein by reference.)

Alternative DNL® Structures

In certain alternative embodiments, DNL® constructs may be formed using alternatively constructed antibodies or antibody fragments, in which an AD moiety may be attached at the C-terminal end of the kappa light chain (Cₖ), instead of the C-terminal end of the Fc on the heavy chain. The alternatively formed DNL® constructs may be prepared as disclosed in Provisional U.S. Patent Application Ser. No. 61/654,310, filed Jun. 1, 2012, 61/662,086, filed Jun. 20, 2012, 61/673,553, filed Jul. 19, 2012, and 61/682,531, filed Aug. 13, 2012, the entire text of each incorporated herein by reference. The light chain conjugated DNL® constructs exhibit enhanced Fc-effector function activity in vitro and improved pharmacokinetics, stability and anti-lymphoma activity in vivo (Rossi et al., 2013, Bioconjug Chem 24:63-71).

Cₖ-conjugated DNL® constructs may be prepared as disclosed in Provisional U.S. Patent Application Ser. No. 61/654,310, 61/662,086, 61/673,553, and 61/682,531. Briefly, Cₖ-AD2-IgG, was generated by recombinant engineering, whereby the AD2 peptide was fused to the C-terminal end of the kappa light chain. Because the natural C-terminus of C_{K} is a cysteine residue, which forms a disulfide bridge to C_{H}l, a 16-amino acid residue “hinge” linker was used to space the AD2 from the C_{K}-V_{H}1 disulfide bridge. The mammalian expression vectors for Cₖ-AD2-IgG-veltuzumab and Cₖ-AD2-IgG-epratuzumab were constructed using the pdHL2 vector, which was used previously for expression of the homologous C_{H}3-AD2-IgG modules. A 2208-bp nucleotide sequence was synthesized comprising the pdHL2 vector sequence ranging from the Bam HI restriction site within the V_{K}/C_{K} intron to the Xho I restriction site 3′ of the Cₖ intron, with the insertion of the coding sequence for the hinge linker (EFPKPSTPPGSSGGAP, SEQ ID NO:14) and AD2, in frame at the 3′end of the coding sequence for C_{K}. This synthetic sequence was inserted into the IgG-pdHL2 expression vectors for veltuzumab and epratuzumab via Bam HI and Xho I restriction sites. Generation of production clones with SpESFX-10 were performed as described for the C_{H}3-AD2-IgG modules. Cₖ-AD2-IgG-veltuzumab and Cₖ-AD2-IgG-epratuzumab were produced by stably-transfected production clones in batch roller bottle culture, and purified from the supernatant fluid in a single step using Mab Select (GE Healthcare) Protein A affinity chromatography.

Following the same DNL® process described previously for 22-(20)-(20) (Rossi et al., 2009, Blood 113:6161-71), Cₖ-AD2-IgG-epratuzumab was conjugated with C_{H}1-DDD2-Fab-veltuzumab, a Fab-based module derived from veltuzumab, to generate the bsHexAb 22*-(20)-(20), where the 22* indicates the Cₖ-AD2 module of epratuzumab and each (20) symbolizes a stabilized dimer of veltuzumab Fab. The properties of 22*-(20)-(20) were compared with those of 22-(20)-(20), the homologous Fc-bsHexAb comprising C_{H}3-AD2-IgG-epratuzumab, which has similar composition and molecular size, but a different architecture.

Following the same DNL® process described previously for 20-2b (Rossi et al., 2009, Blood 114:3864-71), Cₖ-AD2-IgG-veltuzumab, was conjugated with IFNα2b-DDD2, a module of IFNα2b with a DDD2 peptide fused at its C-terminal end, to generate 20*-2b, which comprises veltuzumab with a dimeric IFNα2b fused to each light chain. The properties of 20*-2b were compared with those of 20-2b, which is the homologous Fc-IgG-IFNα.

Each of the bsHexAbs and IgG-IFNα were isolated from the DNL® reaction mixture by MabSelect affinity chromatography. The two Cₖ-derived prototypes, an anti-CD22/CD20 bispecific hexavalent antibody, comprising epratuzumab (anti-CD22) and four Fabs of veltuzumab (anti-CD20), and a CD20-targeting immunocytokine, comprising veltuzumab and four molecules of interferon-α2b, displayed enhanced Fc-effector functions in vitro, as well as improved pharmacokinetics, stability and anti-lymphoma activity in vivo, compared to their Fc-derived counterparts.

Amino Acid Substitutions

In alternative embodiments, the disclosed methods and compositions may involve production and use of proteins or peptides with one or more substituted amino acid residues. For example, the DDD and/or AD sequences used to make DNL® constructs may be modified as discussed above.

The skilled artisan will be aware that, in general, amino acid substitutions typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within ±2 is preferred, within ±1 are more preferred, and within ±0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL website at rockefeller.edu) For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.). Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded protein sequence are well known and a matter of routine experimentation for the skilled artisan, for example by the technique of site-directed mutagenesis or by synthesis and assembly of oligonucleotides encoding an amino acid substitution and splicing into an expression vector construct.

Pre-Targeting

Bispecific or multispecific antibodies may be utilized in pre-targeting techniques. Pre-targeting is a multistep process originally developed to resolve the slow blood clearance of directly targeting antibodies, which contributes to undesirable toxicity to normal tissues such as bone marrow. With pre-targeting, a radionuclide or other therapeutic agent is attached to a small delivery molecule (targetable construct) that is cleared within minutes from the blood. A pre-targeting bispecific or multispecific antibody, which has binding sites for the targetable construct as well as a target antigen, is administered first, free antibody is allowed to clear from circulation and then the targetable construct is administered.

Pre-targeting methods are disclosed, for example, in Goodwin et al., U.S. Pat. No. 4,863,713; Goodwin et al., J. Nucl. Med. 29:226, 1988; Hnatowich et al., J. Nucl. Med. 28:1294, 1987; Oehr et al., J. Nucl. Med. 29:728, 1988; Klibanov et al., J. Nucl. Med. 29:1951, 1988; Sinitsyn et al., J. Nucl. Med. 30:66, 1989; Kalofonos et al., J. Nucl. Med. 31:1791, 1990; Schechter et al., Int. J. Cancer 48:167, 1991; Paganelli et al., Cancer Res. 51:5960, 1991; Paganelli et al., Nucl. Med. Commun. 12:211, 1991; U.S. Pat. No. 5,256,395; Stickney et al., Cancer Res. 51:6650, 1991; Yuan et al., Cancer Res. 51:3119, 1991; U.S. Pat. Nos. 6,077,499; 7,011,812; 7,300,644; 7,074,405; 6,962,702; 7,387,772; 7,052,872; 7,138,103; 6,090,381; 6,472,511; 6,962,702; and 6,962,702, each incorporated herein by reference.

A pre-targeting method of treating or diagnosing a disease or disorder in a subject may be provided by: (1) administering to the subject a bispecific antibody or antibody fragment; (2) optionally administering to the subject a clearing composition, and allowing the composition to clear the antibody from circulation; and (3) administering to the subject the targetable construct, containing one or more chelated or chemically bound therapeutic or diagnostic agents.

Targetable Constructs

In certain embodiments, targetable construct peptides labeled with one or more therapeutic or diagnostic agents for use in pre-targeting may be selected to bind to a bispecific antibody with one or more binding sites for a targetable construct peptide and one or more binding sites for a target antigen associated with a disease or condition. Bispecific antibodies may be used in a pretargeting technique wherein the antibody may be administered first to a subject. Sufficient time may be allowed for the bispecific antibody to bind to a target antigen and for unbound antibody to clear from circulation. Then a targetable construct, such as a labeled peptide, may be administered to the subject and allowed to bind to the bispecific antibody and localize at the diseased cell or tissue.

Such targetable constructs can be of diverse structure and are selected not only for the availability of an antibody or fragment that binds with high affinity to the targetable construct, but also for rapid in vivo clearance when used within the pre-targeting method and bispecific antibodies (bsAb) or multispecific antibodies. Hydrophobic agents are best at eliciting strong immune responses, whereas hydrophilic agents are preferred for rapid in vivo clearance. Thus, a balance between hydrophobic and hydrophilic character is established. This may be accomplished, in part, by using hydrophilic chelating agents to offset the inherent hydrophobicity of many organic moieties. Also, sub-units of the targetable construct may be chosen which have opposite solution properties, for example, peptides, which contain amino acids, some of which are hydrophobic and some of which are hydrophilic.

Peptides having as few as two amino acid residues, preferably two to ten residues, may be used and may also be coupled to other moieties, such as chelating agents. The linker should be a low molecular weight conjugate, preferably having a molecular weight of less than 50,000 daltons, and advantageously less than about 20,000 daltons, 10,000 daltons or 5,000 daltons. More usually, the targetable construct peptide will have four or more residues, such as the peptide DOTA-Phe-Lys(HSG)-Tyr-Lys(HSG)-NH₂ (SEQ ID NO:15), wherein DOTA is 1,4,7,10-tetraazacyclododecane1,4,7,10-tetraacetic acid and HSG is the histamine succinyl glycyl group. Alternatively, DOTA may be replaced by NOTA (1,4,7-triaza-cyclononane-1,4,7-triacetic acid), TETA (p-bromoacetamido-benzyl-tetraethylaminetetraacetic acid), NETA ([2-(4,7-biscarboxymethyl[1,4,7]triazacyclononan-1-yl-ethyl]-2-carbonylmethyl-amino]acetic acid), NODA (1,4,7-triazacylononane-1,4-diacetate) or other known chelating moieties. Chelating moieties may be used, for example, to bind to a therapeutic and or diagnostic radionuclide, paramagnetic ion or contrast agent, such as Al—¹⁸F.

The targetable construct may also comprise unnatural amino acids, e.g., D-amino acids, in the backbone structure to increase the stability of the peptide in vivo. In alternative embodiments, other backbone structures such as those constructed from non-natural amino acids or peptoids may be used.

The peptides used as targetable constructs are conveniently synthesized on an automated peptide synthesizer using a solid-phase support and standard techniques of repetitive orthogonal deprotection and coupling. Free amino groups in the peptide, that are to be used later for conjugation of chelating moieties or other agents, are advantageously blocked with standard protecting groups such as a Boc group, while N-terminal residues may be acetylated to increase serum stability. Such protecting groups are well known to the skilled artisan. See Greene and Wuts Protective Groups in Organic Synthesis, 1999 (John Wiley and Sons, N.Y.). When the peptides are prepared for later use within the bispecific antibody system, they are advantageously cleaved from the resins to generate the corresponding C-terminal amides, in order to inhibit in vivo carboxypeptidase activity. Exemplary methods of peptide synthesis are disclosed in the Examples below.

Where pretargeting with bispecific antibodies is used, the antibody will contain a first binding site for an antigen produced by or associated with a target tissue and a second binding site for a hapten on the targetable construct. Exemplary haptens include, but are not limited to, HSG and In-DTPA. Antibodies raised to the HSG hapten are known (e.g. 679 antibody) and can be easily incorporated into the appropriate bispecific antibody (see, e.g., U.S. Pat. Nos. 6,962,702; 7,138,103 and 7,300,644, incorporated herein by reference with respect to the Examples sections). However, other haptens and antibodies that bind to them are known in the art and may be used, such as In-DTPA and the 734 antibody (e.g., U.S. Pat. No. 7,534,431, the Examples section incorporated herein by reference).

Preparation of Immunoconjugates

In preferred embodiments, a therapeutic or diagnostic agent may be covalently attached to an antibody or antibody fragment to form an immunoconjugate. Where the immunoconjugate is to be administered in concentrated form by subcutaneous, intramuscular or transdermal delivery, the skilled artisan will realize that only non-cytotoxic agents may be conjugated to the antibody. Where a second antibody or fragment thereof is administered by a different route, such as intravenously, either before, simultaneously with or after the subcutaneous, intramuscular or transdermal delivery, then the type of diagnostic or therapeutic agent that may be conjugated to the second antibody or fragment thereof is not so limited, and may comprise any diagnostic or therapeutic agent known in the art, including cytotoxic agents.

In some embodiments, a diagnostic and/or therapeutic agent may be attached to an antibody or fragment thereof via a carrier moiety. Carrier moieties may be attached, for example to reduced SH groups and/or to carbohydrate side chains. A carrier moiety can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995). Alternatively, the carrier moiety can be conjugated via a carbohydrate moiety in the Fc region of the antibody.

Methods for conjugating functional groups to antibodies via an antibody carbohydrate moiety are well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, the Examples section of which is incorporated herein by reference. The general method involves reacting an antibody having an oxidized carbohydrate portion with a carrier polymer that has at least one free amine function. This reaction results in an initial Schiff base (imine) linkage, which can be stabilized by reduction to a secondary amine to form the final conjugate.

The Fc region may be absent if the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); U.S. Pat. Nos. 5,443,953 and 6,254,868, the Examples section of which is incorporated herein by reference. The engineered carbohydrate moiety is used to attach the therapeutic or diagnostic agent.

An alternative method for attaching carrier moieties to a targeting molecule involves use of click chemistry reactions. The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems. Although the copper catalyst is toxic to living cells, the copper-based click chemistry reaction may be used in vitro for immunoconjugate formation.

A copper-free click reaction has been proposed for covalent modification of biomolecules. (See, e.g., Agard et al., 2004, J Am Chem Soc 126:15046-47.) The copper-free reaction uses ring strain in place of the copper catalyst to promote a [3+2] azide-alkyne cycloaddition reaction (Id.). For example, cyclooctyne is an 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions (Id.).

Another type of copper-free click reaction was reported by Ning et al. (2010, Angew Chem Int Ed 49:3065-68), involving strain-promoted alkyne-nitrone cycloaddition. To address the slow rate of the original cyclooctyne reaction, electron-withdrawing groups are attached adjacent to the triple bond (Id.). Examples of such substituted cyclooctynes include difluorinated cyclooctynes, 4-dibenzocyclooctynol and azacyclooctyne (Id.). An alternative copper-free reaction involved strain-promoted alkyne-nitrone cycloaddition to give N-alkylated isoxazolines (Id.). The reaction was reported to have exceptionally fast reaction kinetics and was used in a one-pot three-step protocol for site-specific modification of peptides and proteins (Id.). Nitrones were prepared by the condensation of appropriate aldehydes with N-methylhydroxylamine and the cycloaddition reaction took place in a mixture of acetonitrile and water (Id.). These and other known click chemistry reactions may be used to attach carrier moieties to antibodies in vitro.

Agard et al. (2004, J Am Chem Soc 126:15046-47) demonstrated that a recombinant glycoprotein expressed in CHO cells in the presence of peracetylated N-azidoacetylmannosamine resulted in the bioincorporation of the corresponding N-azidoacetyl sialic acid in the carbohydrates of the glycoprotein. The azido-derivatized glycoprotein reacted specifically with a biotinylated cyclooctyne to form a biotinylated glycoprotein, while control glycoprotein without the azido moiety remained unlabeled (Id.). Laughlin et al. (2008, Science 320:664-667) used a similar technique to metabolically label cell-surface glycans in zebrafish embryos incubated with peracetylated N-azidoacetylgalactosamine. The azido-derivatized glycans reacted with difluorinated cyclooctyne (DIFO) reagents to allow visualization of glycans in vivo.

The Diels-Alder reaction has also been used for in vivo labeling of molecules. Rossin et al. (2010, Angew Chem Int Ed 49:3375-78) reported a 52% yield in vivo between a tumor-localized anti-TAG72 (CC49) antibody carrying a trans-cyclooctene (TCO) reactive moiety and an ¹¹¹In-labeled tetrazine DOTA derivative. The TCO-labeled CC49 antibody was administered to mice bearing colon cancer xenografts, followed 1 day later by injection of ¹¹¹In-labeled tetrazine probe (Id.). The reaction of radiolabeled probe with tumor localized antibody resulted in pronounced radioactivity localization in the tumor, as demonstrated by SPECT imaging of live mice three hours after injection of radiolabeled probe, with a tumor-to-muscle ratio of 13:1 (Id.). The results confirmed the in vivo chemical reaction of the TCO and tetrazine-labeled molecules.

Antibody labeling techniques using biological incorporation of labeling moieties are further disclosed in U.S. Pat. No. 6,953,675 (the Examples section of which is incorporated herein by reference). Such “landscaped” antibodies were prepared to have reactive ketone groups on glycosylated sites. The method involved expressing cells transfected with an expression vector encoding an antibody with one or more N-glycosylation sites in the CH1 or V_{κ} domain in culture medium comprising a ketone derivative of a saccharide or saccharide precursor. Ketone-derivatized saccharides or precursors included N-levulinoyl mannosamine and N-levulinoyl fucose. The landscaped antibodies were subsequently reacted with agents comprising a ketone-reactive moiety, such as hydrazide, hydrazine, hydroxylamino or thiosemicarbazide groups, to form a labeled targeting molecule. Exemplary agents attached to the landscaped antibodies included chelating agents like DTPA, large drug molecules such as doxorubicin-dextran, and acyl-hydrazide containing peptides. The landscaping technique is not limited to producing antibodies comprising ketone moieties, but may be used instead to introduce a click chemistry reactive group, such as a nitrone, an azide or a cyclooctyne, onto an antibody or other biological molecule.

Modifications of click chemistry reactions are suitable for use in vitro or in vivo. Reactive targeting molecule may be formed either by either chemical conjugation or by biological incorporation. The targeting molecule, such as an antibody or antibody fragment, may be activated with an azido moiety, a substituted cyclooctyne or alkyne group, or a nitrone moiety. Where the targeting molecule comprises an azido or nitrone group, the corresponding targetable construct will comprise a substituted cyclooctyne or alkyne group, and vice versa. Such activated molecules may be made by metabolic incorporation in living cells, as discussed above.

Alternatively, methods of chemical conjugation of such moieties to biomolecules are well known in the art, and any such known method may be utilized. General methods of immunoconjugate formation are disclosed, for example, in U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, the Examples section of each incorporated herein by reference.

Therapeutic and Diagnostic Agents

In certain embodiments, the antibodies or fragments thereof may be used in combination with one or more therapeutic and/or diagnostic agents. Where the agent is attached to an antibody or fragment thereof to be administered by subcutaneous, intramuscular or transdermal administration, then only non-cytotoxic agents are contemplated. Non-cytotoxic agents may include, without limitation, immunomodulators, cytokines (and their inhibitors), chemokines (and their inhibitors), tyrosine kinase inhibitors, growth factors, hormones and certain enzymes (i.e., those that do not induce local necrosis), or their inhibitors. Where the agent is co-administered either before, simultaneously with or after the subcutaneous, intramuscular or transdermal antibody formulation, then cytotoxic agents may be utilized. An agent may be administered as an immunoconjugate with a second antibody or fragment thereof, or may be administered as a free agent. The following discussion applies to both cytotoxic and non-cytotoxic agents.

Therapeutic agents may be selected from the group consisting of a radionuclide, an immunomodulator, an anti-angiogenic agent, a cytokine, a chemokine, a growth factor, a hormone, a drug, a prodrug, an enzyme, an oligonucleotide, a pro-apoptotic agent, an interference RNA, a photoactive therapeutic agent, a tyrosine kinase inhibitor, a Bruton kinase inhibitor, a sphingosine inhibitor, a cytotoxic agent, which may be a chemotherapeutic agent or a toxin, and a combination thereof. The drugs of use may possess a pharmaceutical property selected from the group consisting of antimitotic, antikinase, alkylating, antimetabolite, antibiotic, alkaloid, anti-angiogenic, pro-apoptotic agents, and combinations thereof.

Exemplary drugs may include, but are not limited to, 5-fluorouracil, aplidin, azaribine, anastrozole, anthracyclines, bendamustine, bleomycin, bortezomib, bryostatin-1, busulfan, calicheamycin, camptothecin, carboplatin, 10-hydroxycamptothecin, carmustine, celecoxib, chlorambucil, cisplatinum, Cox-2 inhibitors, irinotecan (CPT-11), SN-38, carboplatin, cladribine, camptothecans, cyclophosphamide, cytarabine, dacarbazine, docetaxel, dactinomycin, daunorubicin, doxorubicin, 2-pyrrolinodoxorubicine (2P-DOX), pro-2P-DOX, cyano-morpholino doxorubicin, doxorubicin glucuronide, epirubicin glucuronide, estramustine, epipodophyllotoxin, estrogen receptor binding agents, etoposide (VP16), etoposide glucuronide, etoposide phosphate, floxuridine (FUdR), 3′,5′-O-dioleoyl-FudR (FUdR-dO), fludarabine, flutamide, farnesyl-protein transferase inhibitors, gemcitabine, hydroxyurea, idarubicin, ifosfamide, L-asparaginase, lenolidamide, leucovorin, lomustine, mechlorethamine, melphalan, mercaptopurine, 6-mercaptopurine, methotrexate, mitoxantrone, mithramycin, mitomycin, mitotane, navelbine, nitrosourea, plicomycin, procarbazine, paclitaxel, pentostatin, PSI-341, raloxifene, semustine, streptozocin, tamoxifen, paclitaxel, temazolomide (an aqueous form of DTIC), transplatinum, thalidomide, thioguanine, thiotepa, teniposide, topotecan, uracil mustard, vinorelbine, vinblastine, vincristine and vinca alkaloids.

Toxins may include ricin, abrin, alpha toxin, saporin, ribonuclease (RNase), e.g., onconase, DNase I, Staphylococcal enterotoxin-A, pokeweed antiviral protein, gelonin, diphtheria toxin, Pseudomonas exotoxin, and Pseudomonas endotoxin.

Immunomodulators may be selected from a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β, -λ, or -γ, and stem cell growth factor, such as that designated “Si factor”. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, -λ, and -γ; colony stimulating factors (CSFs) such as macrophage-CSF (M-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-23, IL-25, LIF, kit-ligand or FLT-3, angiostatin, thrombospondin, endostatin, tumor necrosis factor and lymphotoxin.

Chemokines of use include RANTES, MCAF, MIP1-alpha, MIP1-Beta and IP-10.

Radioactive isotopes include, but are not limited to—¹¹¹In, ¹⁷⁷Lu, ²¹²Bi, ²¹³Bi, ²¹¹At, ⁶²Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ²²⁷Th, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, and ²¹¹Pb. The therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, 1-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227 and Fm-255. Decay energies of useful alpha-particle-emitting radionuclides are preferably 2,000-10,000 keV, more preferably 3,000-8,000 keV, and most preferably 4,000-7,000 keV. Additional potential radioisotopes of use include ¹¹C, ¹³N, ¹⁵O, ⁷⁵Br, ¹⁹⁸Au, ²²⁴Ac, ¹²⁶I, ¹³³i, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rb, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ₂₀₁Tl, ₂₂₅Ac, ₂₂₇Th, ₇₆Br, ₁₆₉Yb, and the like.

A variety of tyrosine kinase inhibitors are known in the art and any such known therapeutic agent may be utilized. Exemplary tyrosine kinase inhibitors include, but are not limited to canertinib, dasatinib, erlotinib, gefitinib, imatinib, lapatinib, leflunomide, nilotinib, pazopanib, semaxinib, sorafenib, sunitinib, sutent and vatalanib. A specific class of tyrosine kinase inhibitor is the Bruton tyrosine kinase inhibitor. Bruton tyrosine kinase (Btk) has a well-defined role in B-cell development. Bruton kinase inhibitors include, but are not limited to, PCI-32765 (ibrutinib), PCI-45292, GDC-0834, LFM-A13 and RN486.

Therapeutic agents may include a photoactive agent or dye. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See Joni et al. (eds.), PHOTODYNAMIC THERAPY OF TUMORS AND OTHER DISEASES (Libreria Progetto 1985); van den Bergh, Chem. Britain (1986), 22:430. Moreover, monoclonal antibodies have been coupled with photoactivated dyes for achieving phototherapy. See Mew et al., J. Immunol. (1983), 130:1473; idem., Cancer Res. (1985), 45:4380; Oseroff et al., Proc. Natl. Acad. Sci. USA (1986), 83:8744; idem., Photochem. Photobiol. (1987), 46:83; Hasan et al., Prog. Clin. Biol. Res. (1989), 288:471; Tatsuta et al., Lasers Surg. Med. (1989), 9:422; Pelegrin et al., Cancer (1991), 67:2529.

Corticosteroid hormones can increase the effectiveness of other chemotherapy agents, and consequently, they are frequently used in combination treatments. Prednisone and dexamethasone are examples of corticosteroid hormones.

In certain embodiments, anti-angiogenic agents, such as angiostatin, baculostatin, canstatin, maspin, anti-placenta growth factor (P1GF) peptides and antibodies, anti-vascular growth factor antibodies (such as anti-VEGF and anti-P1GF), anti-Flk-1 antibodies, anti-Flt-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, IP-10, Gro-β, thrombospondin, 2-methoxyoestradiol, proliferin-related protein, carboxiamidotriazole, CM101, Marimastat, pentosan polysulphate, angiopoietin-2, interferon-alpha, interferon-lambda, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline may be of use.

The therapeutic agent may comprise an oligonucleotide, such as a siRNA. The skilled artisan will realize that any siRNA or interference RNA species may be attached to an antibody or fragment thereof for delivery to a targeted tissue. Many siRNA species against a wide variety of targets are known in the art, and any such known siRNA may be utilized in the claimed methods and compositions.

Known siRNA species of potential use include those specific for IKK-gamma (U.S. Pat. No. 7,022,828); VEGF, Flt-1 and Flk-1/KDR (U.S. Pat. No. 7,148,342); Bc12 and EGFR (U.S. Pat. No. 7,541,453); CDC20 (U.S. Pat. No. 7,550,572); transducin (beta)-like 3 (U.S. Pat. No. 7,576,196); KRAS (U.S. Pat. No. 7,576,197); carbonic anhydrase II (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (U.S. Pat. No. 7,781,575), and apolipoprotein B (U.S. Pat. No. 7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Minis Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject DNL® complexes.

Diagnostic agents are preferably selected from the group consisting of a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as ¹⁸F, ⁵²Fe, ¹¹⁰In, ¹¹¹In, ¹⁷⁷Lu, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²Mn, ⁵⁵Co, ⁷²AS, ⁷⁵Br, ⁷⁶Br, ^{82m}Rb, ⁸³Sr, or other gamma-, beta-, or positron-emitters.

Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III).

Ultrasound contrast agents may comprise liposomes, such as gas filled liposomes. Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerytherin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

Methods of Administration

The subject antibodies and immunoglobulins in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active ingredients (i.e., the labeled molecules) are combined in a mixture with one or more pharmaceutically suitable excipients. Sterile phosphate-buffered saline is one example of a pharmaceutically suitable excipient. Other suitable excipients are well known to those in the art. See, e.g., Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The preferred route for administration of the compositions described herein is parenteral injection. Forms of parenteral administration include intravenous, intraarterial, intralymphatic, intrathecal, intraocular, intracerebral, or intracavitary injection. In parenteral administration, the compositions will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hanks' solution. Nonaqueous excipients such as fixed oils and ethyl oleate may also be used. An alternative excipient is 5% dextrose in saline. The excipient may contain minor amounts of additives such as substances that enhance isotonicity and chemical stability, including buffers and preservatives.

Compositions can be presented in unit dosage form, e.g., in ampoules or in multi-dose containers, with an added preservative. Compositions can also take such forms as suspensions, solutions or emulsions in oily or aqueous vehicles, and can contain formulatory agents such as suspending, stabilizing and/or dispersing agents. Alternatively, the compositions can be in powder form for constitution with a suitable vehicle, e.g., sterile pyrogen-free water, before use.

The compositions may be administered in solution. The formulation thereof should be in a solution having a suitable pharmaceutically acceptable buffer such as phosphate, TRIS (hydroxymethyl) aminomethane-HCl or citrate and the like. Buffer concentrations should be in the range of 1 to 100 mM. The formulated solution may also contain a salt, such as sodium chloride or potassium chloride in a concentration of 50 to 150 mM. An effective amount of a stabilizing agent such as mannitol, trehalose, sorbitol, glycerol, albumin, a globulin, a detergent, a gelatin, a protamine or a salt of protamine may also be included.

The dosage of an administered antibody for humans will vary depending upon such factors as the patient's age, weight, height, sex, general medical condition and previous medical history.

Methods of Use

In preferred embodiments, the radiolabeled anti-CD22 antibody or fragment thereof is of use for therapy of cancer. Examples of cancers include, but are not limited to, lymphoma, leukemia and lymphoid malignancies. In preferred embodiments, the antibodies or fragments thereof are of use to treat hematopoietic cancers. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: acute childhood lymphoblastic leukemia, acute lymphoblastic leukemia, acute lymphocytic leukemia, acute myeloid leukemia, adult acute lymphocytic leukemia, adult acute myeloid leukemia, adult Hodgkin's disease, adult Hodgkin's lymphoma, adult lymphocytic leukemia, adult non-Hodgkin's lymphoma, AIDS-related lymphoma, AIDS-related malignancies, central nervous system (primary) lymphoma, central nervous system lymphoma, childhood acute lymphoblastic leukemia, childhood acute myeloid leukemia, childhood Hodgkin's disease, childhood Hodgkin's lymphoma, childhood lymphoblastic leukemia, childhood non-Hodgkin's lymphoma, chronic lymphocytic leukemia, chronic myelogenous leukemia, cutaneous T-cell lymphoma, hairy cell leukemia, Hodgkin's disease, Hodgkin's lymphoma, hypergammaglobulinemia, lymphoproliferative disorders, macroglobulinemia, multiple myeloma, multiple myeloma/plasma cell neoplasm, myelodysplastic syndrome, myelogenous leukemia, myeloid leukemia, myeloproliferative disorders, non-Hodgkin's lymphoma during pregnancy, plasma cell neoplasm/multiple myeloma, primary central nervous system lymphoma, T-cell lymphoma, Waldenstrom's macroglobulinemia, and any other hyperproliferative disease.

The methods and compositions described and claimed herein may be used to detect or treat malignant or premalignant conditions. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976)).

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, and Waldenstrom's macroglobulinemia.

Kits

Various embodiments may concern kits containing components suitable for treating diseased tissue in a patient. Exemplary kits may contain at least one anti-CD22 antibody or fragment thereof, such as epratuzumab, as described herein. A device capable of delivering the kit components by injection, for example, a syringe for subcutaneous injection, may be included. Where transdermal administration is used, a delivery device such as hollow microneedle delivery device may be included in the kit. Exemplary transdermal delivery devices are known in the art, such as 3M's hollow Microstructured Transdermal System (hMTS), and any such known device may be used.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Alternatively, the anti-CD22 antibody or fragment may be delivered and stored as a liquid formulation. Other containers that may be used include, but are not limited to, a pouch, tray, box, tube, or the like. Kit components may be packaged and maintained sterilely within the containers. Another component that can be included is instructions to a person using a kit for its use.

### EXAMPLES

The following examples are provided to illustrate, but not to limit, the claims of the present invention.

The following examples are provided to illustrate, but not to limit, the claims of the present invention.

### Example 1

### 90Y-Labeled Anti-CD22 Antibody (Epratuzumab Tetraxetan) in Refractory/Relapsed Adult CD22+ B-Cell Acute Lymphoblastic Leukemia (ALL)

Summary

Prognosis of patients with relapsed or refractory acute lymphoblastic leukemia is poor and new treatments are needed. A standard 3+3 phase 1 study was performed to assess the feasibility, tolerability, dosimetry and efficacy of yttrium-90-labeled anti-CD22 epratuzumab tetraxetan (⁹⁰Y-DOTA-hLL2) fractionated radioimmunotherapy (RAIT) in adult patients with refractory/relapsed CD22⁺ B− acute lymphoblastic leukemia (ALL). Adults (>18 years) with relapsed or refractory B-cell acute lymphoblastic leukemia (with CD22 expression on at least 70% of blast cells) were enrolled. Patients received one cycle of ⁹⁰Y-DOTA-epratuzumab days 1 and 8 (give or take 2 days), successively at one of four dose levels: 2.5 mCi/m² (92.5 MBq/m², level 1), 5 mCi/m² (185 MBq/m², level 2), 7.5 mCi/m² (277.5 MBq/m², level 3), and 10 mCi/m² (370 MBq/m², level 4). The primary objective was to identify the maximum tolerated dose of ⁹⁰Y-DOTA-epratuzumab. Patients were evaluated for response and minimal residual disease (MRD) between 4 and 6 weeks after RAIT. Flt3-Ligand (Flt3-L) concentration, which may be correlated with the extent of bone marrow aplasia after radiation therapy, was also studied. Seventeen (17) patients (median age: 62 years; range 27-77) were treated (level 1 n=5, level 2 n=3, level 3 n=3, and level 4 n=6). RAIT infusion was overall well-tolerated. One BCR-ABL molecular complete response (CR) was documented at level 2 while 1 CR and 1 CR with incomplete platelets recovery were observed at level 4 (1 Philadelphia (Ph)-positive ALL and 1 Ph-negative ALL) with positive MRD. One dose-limiting toxicity (aplasia lasting 8 weeks) was documented at level 4, but the maximum tolerated dose was not reached. The most common grade 3-4 adverse events were pancytopenia (one at level 2, one at level 3 and six at level 4) and infections (three at level 1, one at level 2 and five at level 4). Two of the 3 responders received a second RAIT cycle. Responses lasted between 7 and 12 months. Interestingly, early increase of Flt3-L concentration seemed predictive of response but not of toxicity.

We conclude that ⁹⁰Y-DOTA-hLL2 RAIT is well-tolerated and induces CR in CD22⁺ relapsed/refractory Ph⁺ or Ph⁻ B-ALL, thus providing a targeted therapy for CD22⁺ B-ALL. A dose of 2×10.0 mCi/m² one week apart/cycle is selected for phase 2 studies.

Patients and Methods

Study Design and Eligibility Criteria—

This prospective Phase-I study was conducted at the CHU of Nantes. Eligibility criteria were: age ≧18 years old, B-ALL with ≧20% of blasts in the bone marrow (BM), CD22+ expression on ≧70% of the blast population, refractory B-ALL defined by treatment failure after 2 successive courses of induction therapy or relapse <6 months after first CR, first relapse or beyond, patients relapsed or refractory to at least one second generation tyrosine kinase inhibitor (TKI) for Philadelphia positive (Ph+) B-ALL, performance status ECOG 0-2, creatinine clearance 50 mL/min, and serum bilirubin 30 μmol/L. Cytologic, immunophenotypic, karyotypic and BCR-ABL (for Ph+B-ALL) molecular analyses were performed on blood samples and/or BM aspirates by standard methods.

Radioimmunotherapy—

Both DOTA-conjugated- and unconjugated epratuzumab were supplied by Immunomedics, Inc. (Morris Plains, N.J.). DOTA-epratuzumab was supplied in 12.0 mg vials (10 mg/mL) for radiolabeling with either ⁹⁰YC1₃ (Ytracis; Cis-Bio International, France) or ¹¹¹InC1₃ (Mallinckrodt Medical B.V., Petten, the Netherlands) according to procedures previously described (Griffiths et al., 2003, J Nucl Med 44:77-84). All patients were to receive one cycle of ⁹⁰Y-DOTA-hLL2 RAIT, according to a standard dose escalation phase 1 trial. It was initially planned to administer 4 infusions of DOTA-epratuzumab (360 mg/m²/day each week during 4 weeks) before RAIT. Only the first two patients received this schedule. Indeed, this “cold phase” was terminated after observing no response in the first two patients, with full saturation of CD22 on the leukemic cells in the first one and rapid epratuzumab clearance for the second one, thus 5 patients were treated at level 1 (3 more patients receiving the RAIT alone). ⁹⁰Y-DOTA-epratuzumab was administered twice on days 1 and 8 (+/−2), successively at 2.5 (92.5 MBq/m², level 1), 5.0 (185 MBq/m², level 2), 7.5 (277.5 MBq/m², level 3), and 10.0 (370 MBq/m², level 4) mCi/m². The protein dose (1.5 mg/kg) was kept constant by co-administration of unlabeled epratuzumab. In the absence of reaction, infusions were completed within 30 min.

Epratuzumab tetraxetan labeled with 3-5 mCi of ¹¹¹Indium was co-infused with the first RAIT injection (day 1), to assess BM tumor targeting and dosimetry (see below). All radioactive materials were handled according to approved protocols at Nantes University Hospital and patients were released when emission at 1 meter was lower than 25 μSv/h, which occurred as soon as day+1. Corticosteroids+polaramine+paracetamol were used as prophylaxis before ⁹⁰Y-DOTA-epratuzumab administration to prevent infusion reactions. Patients in response (CR, CRp or PR, see below) and with no immunization (see below) were allowed to receive a second cycle as consolidation at the same dose level.

Toxicity Evaluation—

A primary objective of the study was to determine the maximum tolerated dose (MTD) of ⁹⁰Y-epratuzumab tetraxetan in adults with refractory/relapsed CD22⁺ B-ALL. Safety was assessed during infusions and regularly after the RAIT over a 6 months period by vital signs, physical examination, serum chemistries, hematology and research of human anti-epratuzumab tetraxetan antibodies (HAHA) by ELISA assay (Immunomedics, Inc) (Morschhauser et al., 2010, J Clin Oncol 28:3709-16). Serious adverse events were documented. Toxicity was determined according to the NCI-CTC criteria version 4. The dose-limiting toxicity (DLT) was defined as any non-reversible grade ≧3 non-hematological toxicity or grade 4 pancytopenia with hypocellular BM lasting for ≧6 weeks. MTD was defined as the dose level at which 2 of 3 or 6 patients experienced a DLT.

Response Assessment—

Responses were evaluated between 4 and 6 weeks after RAIT. However, some patients were evaluated at 15 days because of profound aplasia, to document non-blastic aplasia. Complete response (CR) was defined as <5% marrow blasts, neutrophils ≧1×10⁹/L, platelets ≧100×10⁹/L and no evidence of extramedullary disease. CR with incomplete platelets recovery (CRp) was defined similarly as CR but with platelets counts <100×10⁹/L. Partial response (PR) was defined as a decrease of >50% of BM blasts.

Minimal Residual Disease (MRD)—

MRD was assessed in flow cytometry (FCM) on blood and/or BM samples with an 8 antibodies panel including CD45, CD19, CD10, CD34, CD38, CD58, CD20 and CD22 (FACS CANTOII, BD Biosciences, San Jose, Calif.). Two different anti-CD22 antibodies, clones RFB4 and SHCL-1, were used. RFB4 (PE-conjugated, Invitrogen, Camarillo, Calif.) recognizes the same epitope as epratuzumab and by showing no labeling, confirms the presence of the biologics on the cells. Contrarily, SHCL-1 (PE or PerCp-Cy5.5 conjugated, BD Biosciences) recognizes a different epitope and allows assessing the modulation of CD22 by blasts-cells (Raetz et al., 2008, J Clin Oncol 26:3756-62). MRD was also assessed by RQ-PCR for BCR-ABL in Ph+B-ALL.

Pharmacokinetics, Biodistribution and Dosimetry—

Blood samples for pharmacokinetics were to be obtained before the first RAIT infusion, 5 min before the end of infusion, 5 min after the end of infusion and infusion line washing, then at 1 hour, 2 to 4 hours, 1, 2, 3 to 4 days and 6-7 days after the end of infusion. Pharmacokinetics was also studied after the second infusion with the same blood sample collection schedule. Blood samples were counted using a calibrated γ-counter for ⁹⁰Y and ¹¹¹In activity and the results were corrected for activity decay. Antibody pharmacokinetics was analyzed using compartment analysis software developed in the laboratory. Whole-body anterior and posterior scintigraphy and single-photon emission computed tomography-computerized tomography (SPECT-CT) were recorded between 2 and 4 hours following ¹¹¹In-epratuzumab tetraxetan injection and then at 1, 2, 3 to 5, and 6 to 7 days on a Symbia T (SIEMENS) SPECT/CT γ-camera. Absorbed dose was estimated for lungs, liver, kidneys, spleen using MIRD pamphlet 11 S values adjusted for organ masses and for BM using a method previously described (Ferrer et al., 2012, J Nucl Med Mol Imaging 56:529-37). Organ cumulated activity was calculated using a mono- or bi-exponential model with gunplot software.

Measurement of Blood Biomarker: Flt3-Ligand Concentration—

Fms-like tyrosine kinase 3-Ligand (Flt3-L) blood concentration has been correlated with the extent of BM aplasia after radiotherapy or chemotherapy and during aplastic anemia (Blumenthal et al., 2000, Cancer 88:333-43; Bertho et al., 2001, Int J Radiat Biol 77:703-12). We therefore investigated the serum concentration of this cytokine to evaluate the potential BM toxicity after RAIT for some patients. Serum concentration (pg/mL) was evaluated using ELISA (R&D Systems, DY308) before and at various intervals after RAIT. Samples were analysed in duplicate and data are expressed as mean±SD.

Results

Patient Characteristics—

Over a three year period, 20 patients were enrolled. Three patients were not considered for evaluation because of progression (n=2) or non-blastic aplasia (n=1) before RAIT. Overall, 17 patients (male n=10; median age: 62 years, range: 27-77) were treated (5 at level 1 including 2 previously treated with cold epratuzumab, 3 at level 2, 3 at level 3, and 6 at level 4). Demographics of treated patients are given in Table 1.

Toxicity—

The salvage regimen was overall well-tolerated, since almost all grade 3/4 toxicities were expected pancytopenia. Five patients presented reactions (3 grade 1, 1 grade 2 and 1 grade 3 in a patient with a previous history of severe allergic reactions) during the first RAIT infusion, preventing administration of the entire dose for two patients. However, all patients received a second injection for which the administration rate was reduced and tolerance was improved as no toxicity occurred. At level 4, all of the 6 patients but one presented with reversible grade 4 hematologic toxicities followed by progression or CR. Indeed, one DLT was documented at level 4 (non-blastic aplasia resolving after 8 weeks), but MTD was not reached. No grade 3-4 hepatic or renal toxicities and no toxic death were observed. All patients were examined for detection of human anti-hLL2 antibodies (HAHA) before RAIT (n=17) and before the second injection (n=17), while 11 patients were examined after the two injections (at day+15 n=2; at day+30 n=6, at day+45 n=2). Also the three responders were examined at 3 (n=3) and 6 (n=3) months post-RAIT. None of the patients developed HAHA.

TABLE 1
Demographics of patients included and treated (n = 17).

| Patients | n = 17 | |
|---|---|---|
| Gender: male | 10 | (59%) |
| Median age: years (range) | 62 | (27-77) |
| <55 years | 5 | (29%) |
| Status | | |
| First relapse | 10 (including 4 | |
| | refractory relapses) | |
| Second relapse | 3 | |
| Third relapse | 2 | |
| Primary refractory | 2 | |
| Previous allotransplant | 4 | (23.5%) |
| Median white blood count at time of | 3 (0.2-218) × 10⁹/L | |
| inclusion* | | |
| Median % of peripheral blasts | 8 | (0-99.5) |
| Median % of CD22-RFB4 expression | 100 | (93-100) |
| Median % of CD22-SHCL-1 expression | 100 | (93-100) |
| Karyotype | | |
| t(9; 22) | 6 | |
| Hyperdiploidy | 1 | |
| Hypodiploidy | 1 | |
| Near-triploidy | 1 | |
| MLL rearrangement | 1 | |
| Del4q (+Ikaros mutation) | 1 | |
| Complex | 3 | |
| Normal (+Ikaros mutation) | 1 | |
| Unknown | 2 | |
| Median % of blasts in bone marrow at | 75 | (15.5-98.5) |
| time of inclusion* | | |
| Median % of CD22-RFB4 expression | 100 | (41-100) |
| Median % of CD22-SHCL-1 expression | 100 | (90-100) |
| Median interval between diagnosis and | 16.5 | (1-101) |
| salvage chemoimmunotherapy: months | | |
| (range) | | |

*The median percentage of BM blasts was 75% vs. 8% in blood (n = 11 patients). The blast population was 100% CD22-SHCL-1- and CD22-RFB4-positive in blood and BM in all but two patients (93/93% in blood and 90/93% in BM with SHCL-1 and RFB4 respectively in one patient, 100/41% with SHCL-1 and RFB4 in BM with no peripheral blasts in the other).

Interestingly, one patient had already received epratuzumab as part of another trial (NCT01219816) three months before. He obtained a partial response with 100% of SHCL-1+ blasts but 0% of RFB4 binding, suggesting a persistent targeting of epratuzumab to BM blasts without loss of the CD22/epratuzumab complex from the cell surface. Two more months were necessary to document the elimination of epratuzumab from the blast surface, by demonstrating 100/100% of SHCL-1 and RFB4 labeling, to finally include the patient in this trial.

Responses and Survival—

Two patients reached CR at 5 weeks (level 4) and 6 weeks (level 2) (Chevallier et al., 2013, Eur J Haematol 91:552-6) of RAIT and 1 patient obtained CRp at 8 weeks (level 4). No response was seen at levels 1 and 3. Two responders (1 level 2 and 1 level 4) received a second cycle at the same dose level. At the time of analysis, all patients have died from disease progression except one responder (alive at +9 months) and a non-responder with Ph+B-ALL (alive at +27 months). Outcomes of responders are described in Example 2.

MRD Analysis—

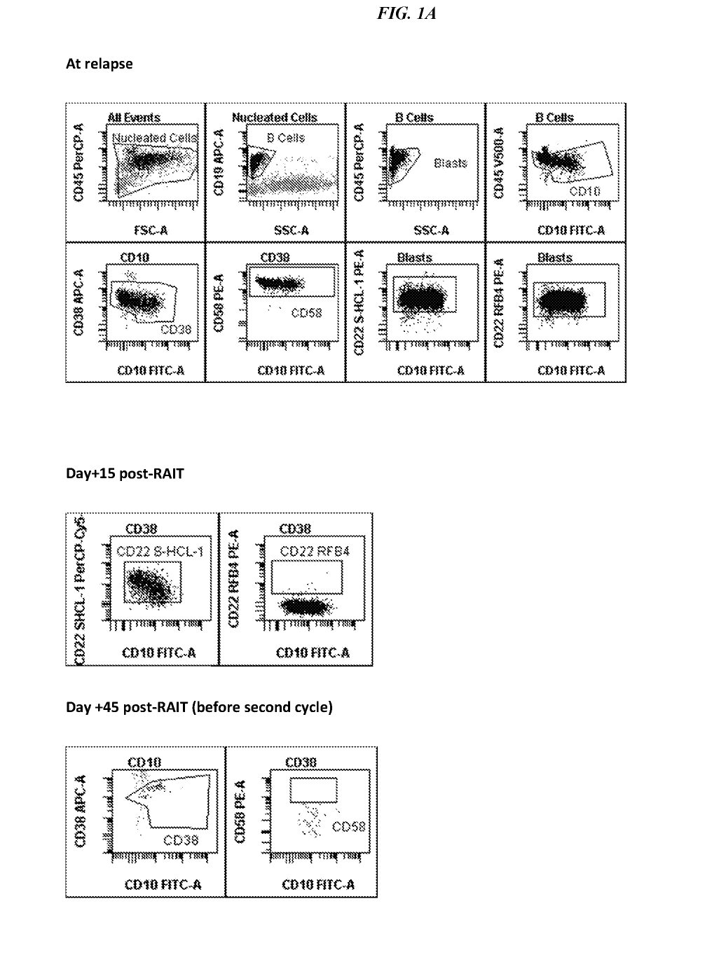

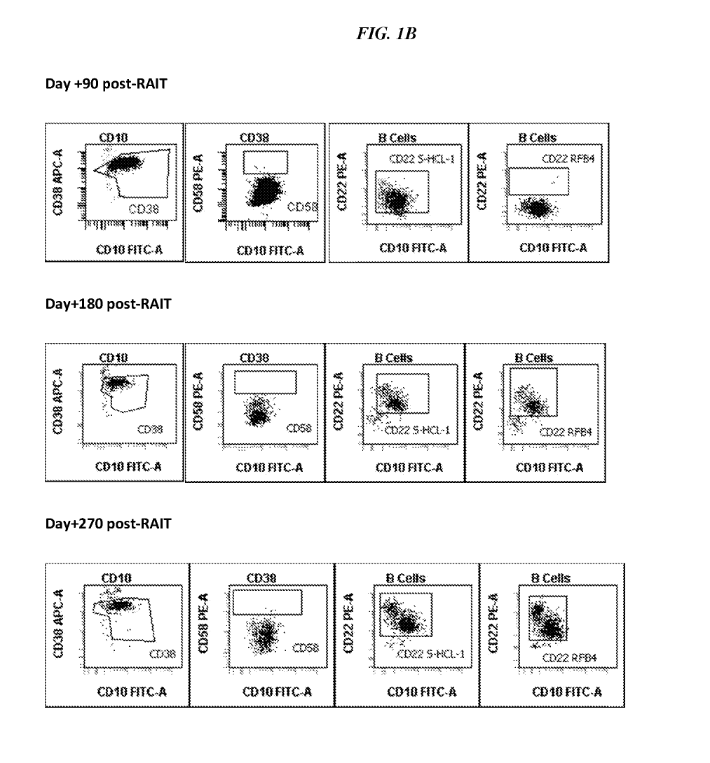

Among the 14 non-responders, 8 were evaluated by FCM to assess the binding of epratuzumab on residual blast population at the time of response assessment. Three profiles were observed. Four patients showed 100% RFB-4 and SHCL-1 positivity suggesting a loss or absence of binding of epratuzumab. Three patients had 100% SHCL-1 positivity but no labelling with RFB4, suggesting a persistent targeting of epratuzumab without loss of the CD22/epratuzumab complex. Finally, for one patient, SHCL-1 and RFB4 positivity was respectively 84% and 75%, suggesting a partial targeting of epratuzumab and partial internalisation of the CD22/epratuzumab complex, loss of CD22 expression or reappearance of a new blast population together with the persistence of that which had contact with the biologic. Among responders, the level 2 patient obtained negative BM immunophenotypic and molecular MRD at the time of CR (6 weeks from RAIT). Immunophenotypic MRD persisted at 3, 6 and 9 months while BCR-ABL transcript was detected again at 9 months, predicting the relapse which occurred at 12 months (FIG. 1A-1B). Follow-up of MRD for the two other responders is given in Example 2.

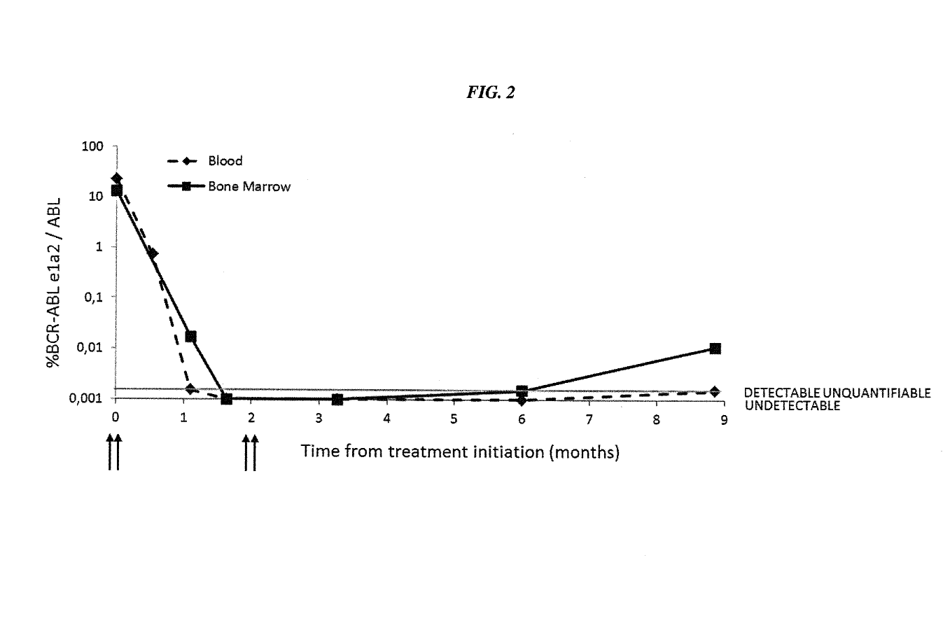

FIG. 1A at Day 15 post-RAIT showed positive MRD at 2.7%, showing a 100% CD22 SHCL-1 blast expression but a 100% reduction of RFB4− binding, suggesting persistent targeting of epratuzumab to bone marrow leukemic blasts without loss of the CD22/epratuzumab complex from the cell surface. FIG. 1A at day 45 post-RAIT showed negative MRD (<5 10⁻⁵). FIG. 1B at day 90 post-RAIT showed MRD is still negative (<10⁻⁵). A large proportion of hematogones (CD19+/CD10+/CD38+) was detected at day+90. Normal bone marrow B cells showed a 100% CD22 SHCL-1 expression but a 100% reduction of RFB4− binding, suggesting a persistent targeting of epratuzumab on normal cells. FIG. 1B at day 180 post-RAIT showed MRD still negative (<10⁻⁵), and a large proportion of hematogones (CD19+/CD10+/CD38+) remains. Normal bone marrow B-cells are 100% positive for both SHCL-1 and RFB4, demonstrating the disappearance of epratuzumab from the cell surface. FIG. 1B at day 279 post-RAIT showed that the same results as day+180 was observed at 9 months from the RAIT day+1. Unfortunately the patient relapsed at +12 months. FIG. 2 shows a follow-up of molecular minimal residual disease (MRD) after radioimmunotherapy (RAIT) cycle 1 $2 (arrows) in the bone marrow of the patient achieving complete remission at level 2. The patient relapsed at 12 months.

Pharmacokinetics, Imaging and Dosimetry—

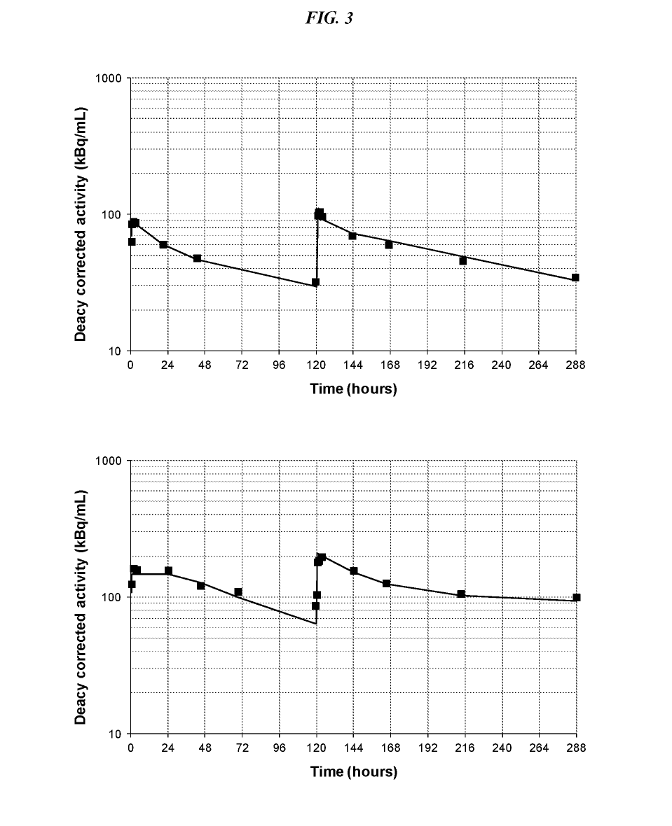

Pharmacokinetics of the antibody was monitored from the first infusion of a mixture of ¹¹¹In and ⁹⁰Y-labeled epratuzumab up to 7 days after the second infusion of ⁹⁰Y-labeled epratuzumab. Pharmacokinetics was assessed for the first infusion only for 5 patients and in 11 cases for the two infusions. A major finding was that in 9 of the 16 patients, the pharmacokinetics of the antibody could not be represented by a classical exponential concentration decay. By contrast, the kinetics observed after the second infusion were well fitted using two exponentials (FIG. 3). The first two patients who had received unlabeled antibody as a pre-dose did not show this effect.

There was no major difference between ¹¹¹In and ⁹⁰Y data. Thus the kinetics of the antibody were studied using the ⁹⁰Y data, which allowed the two infusions to be considered together, in terms of activities corrected to the time of infusion, thus reflecting the protein pharmacokinetics.

The first curve corresponds to patient 4 who received 210 GBq in the first infusion and 212 GBq in the second, 5 days later. The second curve corresponds to patient 3 who received 175 GBq in the first infusion and 175 GBq in the second, 5 days later. Note the shape of the blood activity curve after the first infusion in patient 3.

Of note, for 5 patients, blood and serum were counted separately showing parallel kinetics, excluding any significant activity binding to circulating cells. Studies are in progress to try and understand the phenomenon and to develop a pharmacokinetic model. No obvious correlation with blast numbers or targeting to bone marrow was found so far. Altogether, there was a large variability from one patient to another as shown by plasma clearance, which ranged from 1.6 mL/hr to 128 mL/hr with a mean of 35 mL/hr. Thus clearance in some patients was higher than the typical value of 10 mL/hr, suggesting antigen-induced antibody clearance.

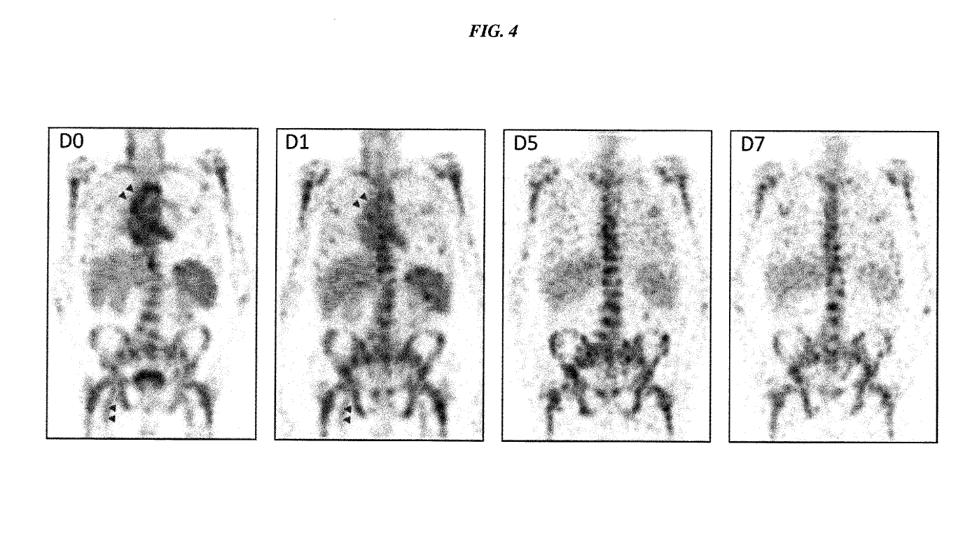

Biodistribution of ¹¹¹In-epratuzumab tetraxetan was studied in 15 patients: 5/5 at level 1, 1/3 at level 2, 2/3 at level 3 and 6/6 at level 4. As shown in FIG. 4, all presented an expected uptake on potential disease sites (blood, spleen, liver, and BM). Individual dosimetry was performed in 11 patients, altered general condition preventing to complete the planned imaging protocol in 4 patients.

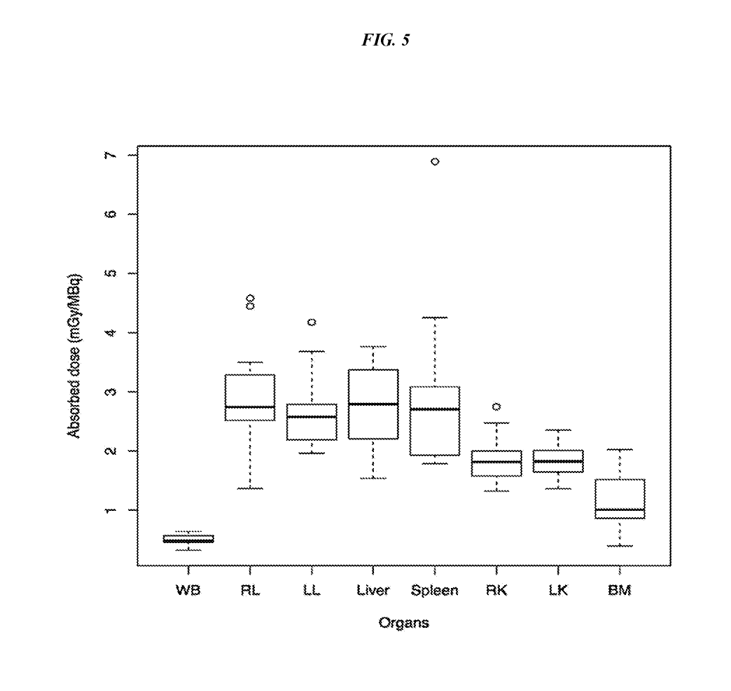

BM and organ dosimetry from all studied patients are presented in FIG. 5 and expressed as absorbed dose per unit of activity to allow comparison. The highest median absorbed doses were observed at level 4, with median of 3447 mGy (4168-2910), 3123 mGy (3996-2359) 3385 mGy (4892-2575) and 2705 mGy (2852-1789 mGy) for liver, lungs, spleen and kidneys respectively. Interestingly, the highest individual spleen absorbed dose was observed at level 3 (5416 mGy) probably reflecting disease′infiltration. At level 4, median BM absorbed dose were 1820 mGy, estimated at 1283 and 2663 mGy in the 2 responders and 1361 and 2280 mGy, in the 2 non-reponders, the patient exhibiting DLT presenting the lowest value. At level 4, the 2 responders had higher spleen absorbed doses (3524 and 4892 mGy) than the 2 non-responders (2576 and 3246 mGy).

Flt3-Ligand—

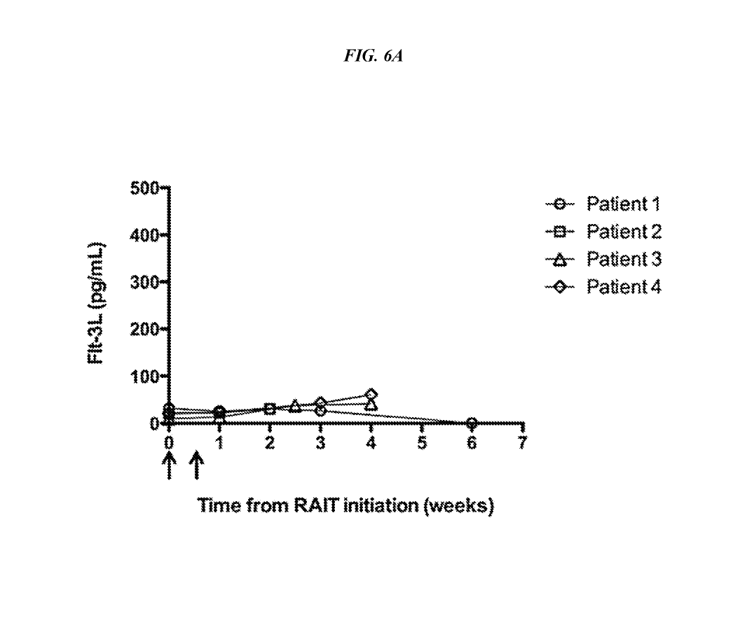

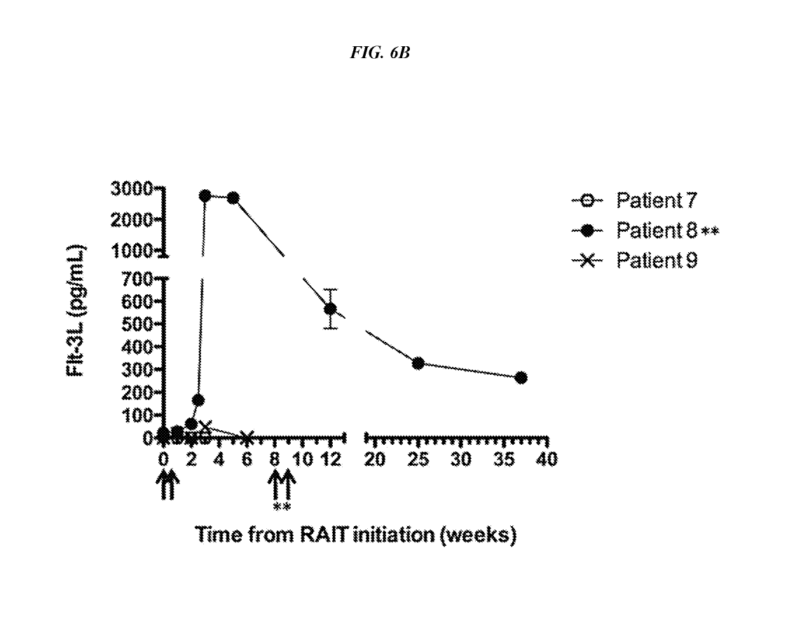

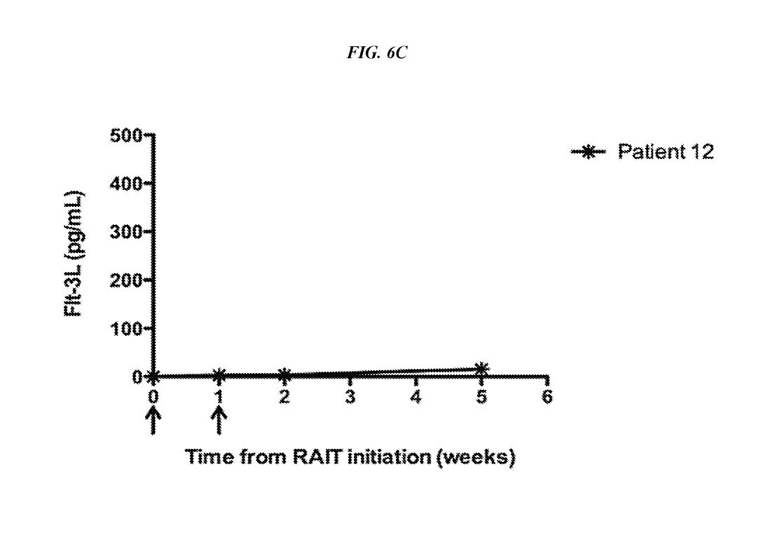

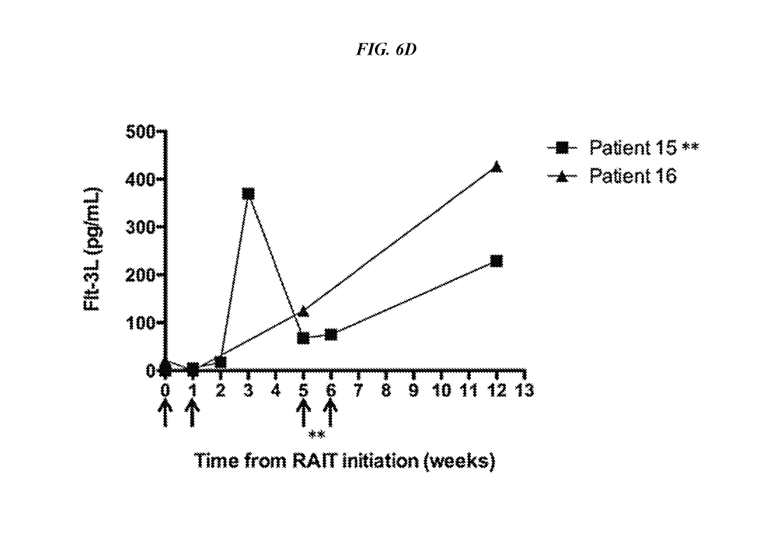

Nine patients were evaluated for Flt3-L concentrations at various times after RAIT: 4/5 at level 1, 3/3 at level 2, 1/3 at level 3 and 2/6 (the 2 patients in CR) at level 4 (FIG. 6A-D). Flt3-L was never detected at baseline and no increase was seen between RAIT and response evaluation for non-responders, suggesting no correlation between this parameter and the procedure's toxicity. Conversely, early significant increases of Flt3-L concentrations were observed in at least two responders while the patients were still in aplasia, suggesting that Flt3-L concentration is predictive of response after RAIT. The highest Flt3-L concentration (2755 pg/mL) was observed in the level 2 responder, who had also the longest remission.

As observed for level 2, patient 15 showed increased Flt3-L concentration 3 weeks after treatment initiation and before documentation of CR (day+34) (FIG. 6D). Contrary to the two other responders (patient 8 level 2 and patient 16 level 4, FIGS. 6B and 6D), where Flt3-L concentration remained high at the time of CR documentation, Flt3-L concentration returns under 100 pg/mL in this patient. A second RAIT cycle (**) was started 5 weeks after treatment initiation in this patient. It was again associated with increased Flt3-L concentration (12 weeks after treatment initiation).

Similarly, patient 16 showed an increased Flt3-L concentration 5 weeks after treatment initiation before documentation of CR with incomplete platelets recovery (CRp, day+62, aplasia lasted 8 weeks) (FIG. 6D). Unfortunately, no sample was available in this patient within the 4 weeks following RAIT, at a time where the early and transient Flt3-L increases were documented for the other responders. The Flt3-L concentration remained high (12 weeks after treatment initiation) between documentation of CRp and platelets recovery (>100×10⁹/L, day+107). Patient 16 refused a second cycle of RAIT.

Discussion

This Example reports the results of a phase 1 study using ⁹⁰Yttrium labeled anti-CD22 (epratuzumab tetraxetan) RAIT for the treatment of relapsed/refractory CD22+ B-ALL. It demonstrates not only the feasibility but also the safety and efficacy of the approach, since three CRs were obtained, including one molecular BCR-ABL response in a patient with Ph+ CD22+B-ALL. Moreover, two responders received a consolidation treatment without toxicity. In fact, this is the first demonstration of the efficacy of RAIT in ALL and the results are very promising considering the dismal prognosis of relapsed B-ALL (Thomas et al., 1999, Cancer 86:1216-30; Tavernier et al., 2007, Leukemia 21:1907-14; Fielding et al., 2007, Blood 109:944-50; Oriol et al., 2010, Haematologica 95:589-96; Gokbuget et al., 2012, Blood 120:2032-41).

Radiation exposure was minimal for the patient and environment, allowing ambulatory RAIT with no need to isolate the patient after treatment. One DLT was observed at 2×370 MBq/m² (level 4), but MTD was not reached. BM absorbed doses were unable to predict severity of hematologic toxicity as the patient exhibiting DLT presented the lowest BM absorbed dose of level 4 patients. No extra-hematologic radiation related toxicity occurred. Organs normalized absorbed doses remained in the same range as those previously reported (Sharkey et al., 2003, J Nucl Med 44:2000-18), and below safety recommendations (Milano et al., 2008, Radiat Oncol 3:36). The 2 responders at level 4 showed widely different BM absorbed doses. As previously reported (Sharkey et al., 2003, J Nucl Med 44:2000-18), dosimetry alone (in particular BM dose in this study) is not sufficient to predict either toxicity or efficacy, particularly in such study enrolling a few patients with various prior treatment regimens and tumor spread. Thus, a better understanding of resistance mechanisms to such an approach is needed. Binding of the antibody to leukemic blasts could be defective in some patients, as suggested by retained positivity of blasts with both RFB4 and SHCL-1 immunophenotyping antibodies after RAIT. The presence of blasts negative with both RFB4 and SHCL-1 suggests internalization of the CD22/epratuzumab complex, or a partial loss of CD22 expression. However, for other non-responders as well as two responders, persistence of the CD22/epratuzumab complex on blasts was observed for several months after RAIT. Thus, inherent or acquired resistance to radiation may be implicated in relation to possible specific immunophenotypic features, for example CD24 expression (Uckun et al., 1993, Blood 81:1323-32), or molecular characteristics of the blasts (for example over expression of Bcl-x1) (Findley et al., 1997, Blood 89:2986-93). As a consequence, radiosensitizers could be of interest to improve the results of the RAIT for B-ALL patients (Han et al., 2013, Neoplasia 15:1207-17).

Also, ALL cells being mostly disseminated in bone marrow, either isolated or as microscopic clusters, replacement of beta-emitters, such as ⁹⁰Y used here, which have relatively long path lengths (around 10 mm), should be considered. Radionuclides emitting high-linear energy transfer alpha particles have been demonstrated to be more toxic to isolated target cells and cell toxicity is achieved with only a few disintegrations at the cell surface (Barbet et al., 2012, Methods Mol Biol 907:681-97). However, clinical experience with these a-emitting radionuclides remains limited to feasibility studies and ongoing clinical trials.

Surprisingly, early increase of Flt3-L concentration was predictive of response in this study. This cytokine is expressed by normal stromal cells and is considered to play an important role in the regulation of hematopoiesis. Flt3-L blood concentration has been shown to be inversely correlated with the degree and extent of BM aplasia after radiotherapy or chemotherapy and during aplastic anemia (Blumenthal et al., 2000, Cancer 88:333-43; Bertho et al., 2001, Int J Radiat Biol 77:703-12).

Here, the absence of increase of Flt3-L concentration in non-responders could be due to the persistence of BM leukemic cells. Indeed, it may be emphasized that the disappearance of BM blasts allows the restoration of normal production of Flt3-L associated with normal hematopoiesis. Future studies should investigate the predictive value of this factor early after chemotherapy, radiotherapy or allo-SCT in hematologic diseases.

A moderate immune thrombocytopenic purpura appeared after a second RAIT cycle in a patient of this study. Other specific adverse reactions may also occur after RAIT, for example immunization or secondary myelodysplastic syndrome (MDS)/acute myelogenous leukemia (AML). Immunization seems to be a major problem only in previously untreated patients (Juweid et al., 2002, J Nucl Med 43:1507-29). Humanization of antibodies reduces this risk and no HAHA was detected here, as already reported in a previous study testing fractionated ⁹⁰Y-epratuzumab tetraxetan (Morschhauser et al., 2010, J Clin Oncol 28:3709-16), confirming its very low immunogenicity. Regarding the long-term occurrence of MDS/AML after RAIT, the updated results of the randomized phase III FIT study assessing ⁹⁰Y-ibritumomab tiuxetan as consolidation in first line therapy in follicular lymphoma patients, with a median follow-up of 7.3 years, showed a significant higher 0.5% incidence rate of secondary MDS/AML in the RAIT group compared to the control group (0.07% only, p=0.042) (Morschhauser et al., 2013, J Clin Oncol 31:1977-83). This incidence is however very low and this risk has to be balanced with the risk of ALL itself if RAIT is used as a salvage regimen in patients, as done here.

In conclusion, ⁹⁰Y-epratuzumab tetraxetan RAIT represents an innovative therapy for relapsed/refractory CD22+Ph+ or Ph− B-ALL and increases the therapeutic armamentarium for these patients for whom outcome remains unfavourable. Such a strategy could also provide a bridge allowing more patients to receive allo-SCT after achieving CR. Phase-II studies should be initiated at the recommended dose of 10 mCi/m² (370 MBq/m²) given twice, one week apart/cycle.

### Example 2

### Outcomes of RAIT Responders

The RAIT responder at level 2 was a 57-year old woman in third relapse of Ph⁺ B-ALL. She had normal levels of hemoglobin, leukocytes and platelets, with no peripheral blasts. BM showed 60% blast infiltration, and BCR-ABL/ABL ratios were 22.4% and 13% in the blood and BM respectively. At day+15, she had non-blastic aplasia and was documented with immunophenotypic and molecular CR (FIG. 1A). This patient received a second RAIT cycle (2 infusions at the same dose level) at 8 weeks from the first. A moderate immune thrombocytopenic purpura occurred with spontaneous favorable evolution. This observation has already been published (Chevallier et al., 2013, Eur J Haematol 91:552-56). This patient relapsed at 12 months from the first cycle and rapidly died from progression.

At level 4, the first responder was a 77-year old man in first relapse of a Ph⁺ B-ALL, previously treated with imatinib and dasatinib. He had 50% circulating blasts, BM showed 95% blast infiltration, and BCR-ABL/ABL ratios in blood and BM were 129% and 116%, respectively. CR was reached at 5 weeks from RAIT but with positive MRD. He received a second RAIT cycle at the same level at 7 weeks, relapsed at 7 months from the first RAIT and is still alive, receiving blinatumomab as part of another trial.

The second responder at level 4 was a 44-year old B-ALL patient (karyotype unknown), refractory to induction. Because of heavy antecedents (Fallot tetralogy, vascular cerebral accident, primary hemochromatosis), he was proposed to enter the trial. He had 18% of circulating blasts and 75% BM blast infiltration. After RAIT, he required treatment for aspergillosis and cardiogenic pulmonary oedema, which may explain the long subsequent delay resulting in CRp only at 8 weeks. Platelets recovery was achieved at day+107. He refused the second cycle, relapsed at 7 months from RAIT and died rapidly thereafter.

None of the responders were consolidated by allo-SCT. The level 2 responder refused allo-SCT despite a compatible donor while the two patients at level 4 were not eligible for allo-SCT due to older age or bad performance status.

### Example 3

### Follow-Up of MRD for the Two Responders at Level 4

The older responder at level 4 retained positive immunophenotypic MRD (blood: 0.05%, BM: 0.9%) with RFB4 negativity on the remaining SHCL-1⁺ blasts. Molecular MRD was also positive at 1.4% in blood and 1.8% in BM. At 3 months, after a second RAIT cycle, immunophenotypic MRD was at 0.004% in the blood still with RFB4 negativity, and molecular MRD was at 0.18%. At relapse the 14% of BM blasts were both SHCL-1 and RFB4 positive (loss of epratuzumab), and BCR-ABL/ABL blood ratio was 35%.

The younger responder at level 4 had 1.77% immunophenotypic BM MRD (SHCL-1⁺/RFB4⁻). At 3 months and with no second RAIT, BM immunophenotypic MRD decreased to 0.007% but with RFB4 positivity, suggesting loss of epratuzumab. Blood immunophenotypic MRD was 0.01% at 3 months and 0.11% at 4 months. At relapse, the 92% of BM blasts were SHCL-1⁺/RFB4⁺.

What is claimed is:

1. A method of treating refractory and/or relapsed B-cell acute lymphoblastic leukemia (ALL), comprising administering to a patient with refractory/relapsed B-cell ALL a ⁹⁰Y-labeled anti-CD22 antibody.

2. The method of claim 1, wherein the ⁹⁰Y-labeled anti-CD22 antibody is epratuzumab tetraxetan.

3. The method of claim 1, wherein the patient is refractory to treatment with at least one prior therapy.

4. The method of claim 1, wherein the patient is refractory to treatment with an agent selected from the group consisting of vincristine, dexamethasone, prednisone, doxorubicin, daunorubicin, cyclophosphamide, L-asparaginase, etoposide, methotrexate, 6-mercaptopurine, a tyrosine kinase inhibitor and radiation therapy.

5. The method of claim 4, wherein the tyrosine kinase inhibitor is selected from the group consisting of imatinib, dasatinib, nilotinib, bosutinib and ponatinib.

6. The method of claim 1, wherein the patient is resistant/relapsed to multiple therapies.

7. The method of claim 1, wherein the patient is positive for Philadelphia chromosome (BCR-ABL).

8. The method of claim 1, wherein the humanized anti-CD22 antibody or fragment thereof comprises the light chain complementarity determining region (CDR) sequences CDR1 (KSSQSVLYSANHKYLA, SEQ ID NO:16), CDR2 (WASTRES, SEQ ID NO:17), and CDR3 (HQYLSSWTF, SEQ ID NO:18) and the heavy chain CDR sequences CDR1 (SYWLH, SEQ ID NO:19), CDR2 (YINPRNDYTEYNQNFKD, SEQ ID NO:20), and CDR3 (RDITTFY, SEQ ID NO:21).

9. The method of claim 1, wherein the ⁹⁰Y-labeled anti-CD22 antibody is administered at a dose of about 100, 200, 300, or 400 MBq/m².

10. The method of claim 1, wherein the ⁹⁰Y-labeled anti-CD22 antibody is administered at a dose of between 90 and 400 MBq/m².

11. The method of claim 1, wherein the ⁹⁰Y-labeled anti-CD22 antibody is administered at a dose of between 2×370 MBq/m² one week apart per cycle.

12. The method of claim 1, wherein the ⁹⁰Y-labeled anti-CD22 antibody is administered twice on days 1 and 8 of therapy,

13. The method of claim 1, wherein the ⁹⁰Y-labeled anti-CD22 antibody does not induce a dose-limiting toxicity.

14. The method of claim 1, wherein therapy with ⁹⁰Y-labeled anti-CD22 antibody is capable of inducing a complete response in the patient.

15. The method of claim 1, wherein therapy results in a response lasting between 7 and 12 months.

16. The method of claim 1, wherein an increase in serum Flt3-L concentration is predictive of therapeutic response, but not toxicity.

17. The method of claim 1, wherein the ⁹⁰Y is attached to a DOTA moiety on the antibody or fragment thereof.

18. The method of claim 1, further comprising administering a radiosensitizing agent to the patient.

19. The method of claim 1, wherein the anti-CD22 antibody is RFB4.


SEQUENCE LISTING (21 sequences)

SEQ ID NO: 1 - protein, 44 aa, Artificial Sequence
  Description of Artificial Sequence Synthetic polypeptide
SHIQIPPGLTELLQGYTVEVLRQQPPDLVEFAVEYFTRLREARA

SEQ ID NO: 2 - protein, 45 aa, Artificial Sequence
  Description of Artificial Sequence Synthetic polypeptide
CGHIQIPPGLTELLQGYTVEVLRQQPPDLVEFAVEYFTRLREARA

SEQ ID NO: 3 - protein, 17 aa, Artificial Sequence
  Description of Artificial Sequence Synthetic peptide
QIEYLAKQIVDNAIQQA

SEQ ID NO: 4 - protein, 21 aa, Artificial Sequence
  Description of Artificial Sequence Synthetic peptide
CGQIEYLAKQIVDNAIQQAGC

SEQ ID NO: 5 - protein, 50 aa, Artificial Sequence
  Description of Artificial Sequence Synthetic polypeptide
SLRECELYVQKHNIQALLKDSIVQLCTARPERPMAFLREYFERLEKEEAK

SEQ ID NO: 6 - protein, 55 aa, Artificial Sequence
  Description of Artificial Sequence Synthetic polypeptide
MSCGGSLRECELYVQKHNIQALLKDSIVQLCTARPERPMAFLREYFERLEKEEAK

SEQ ID NO: 7 - protein, 23 aa, Artificial Sequence
  Description of Artificial Sequence Synthetic peptide
CGFEELAWKIAKMIWSDVFQQGC

SEQ ID NO: 8 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 51 aa, Artificial Sequence
  Description of Artificial Sequence Synthetic polypeptide
SLRECELYVQKHNIQALLKDVSIVQLCTARPERPMAFLREYFEKLEKEEAK

SEQ ID NO: 11 - protein, 54 aa, Artificial Sequence
  Description of Artificial Sequence Synthetic polypeptide
SLKGCELYVQLHGIQQVLKDCIVHLCISKPERPMKFLREHFEKLEKEENRQILA

SEQ ID NO: 12 - protein, 44 aa, Artificial Sequence
  Description of Artificial Sequence Synthetic polypeptide
SHIQIPPGLTELLQGYTVEVGQQPPDLVDFAVEYFTRLREARRQ

SEQ ID NO: 13 - protein, 44 aa, Artificial Sequence
  Description of Artificial Sequence Synthetic polypeptide
SIEIPAGLTELLQGFTVEVLRHQPADLLEFALQHFTRLQQENER

SEQ ID NO: 14 - protein, 16 aa, Artificial Sequence
  Description of Artificial Sequence Synthetic peptide
EFPKPSTPPGSSGGAP

SEQ ID NO: 15 - protein, 4 aa, Artificial Sequence
  Description of Artificial Sequence Synthetic peptide
FKYK

SEQ ID NO: 16 - protein, 16 aa, Artificial Sequence
  Description of Artificial Sequence Synthetic peptide
KSSQSVLYSANHKYLA

SEQ ID NO: 17 - protein, 7 aa, Artificial Sequence
  Description of Artificial Sequence Synthetic peptide
WASTRES

SEQ ID NO: 18 - protein, 9 aa, Artificial Sequence
  Description of Artificial Sequence Synthetic peptide
HQYLSSWTF

SEQ ID NO: 19 - protein, 5 aa, Artificial Sequence
  Description of Artificial Sequence Synthetic peptide
SYWLH

SEQ ID NO: 20 - protein, 17 aa, Artificial Sequence
  Description of Artificial Sequence Synthetic peptide
YINPRNDYTEYNQNFKD

SEQ ID NO: 21 - protein, 7 aa, Artificial Sequence
  Description of Artificial Sequence Synthetic peptide
RDITTFY